(19) United States
(12) Patent Application Publication
(10) Pub. No.: US 2025/0025479 A1
(43) Pub. Date: Jan. 23, 2025

(54) TRANSPORE DELIVERY OF STEROIDS AND LARGE MOLECULES

(71) Applicant: Joel STUDIN, Roslyn, NY (US)

(72) Inventor: Joel STUDIN, Roslyn, NY (US)

(21) Appl. No.: 18/581,836

(22) Filed: Feb. 20, 2024

Related U.S. Application Data
(63) Continuation of application No. 18/336,163, filed on Jun. 16, 2023, now abandoned.
Application No. 18/336,163 is a continuation of application No. 18/046,350, filed on Oct. 13, 2022, now abandoned.
Application No. 18/046,350 is a continuation of application No. 16/586,406, filed on Sep. 27, 2019, now abandoned.
(60) Provisional application No. 62/738,736, filed on Sep. 28, 2018.

(51) Int. Cl. A61K 31/58; A61K 9/00; A61K 9/06; A61K 31/167; A61K 31/245; A61K 31/381; A61K 31/435; A61K 31/451; A61K 31/4706; A61K 31/5375; A61K 31/575; A61K 47/08; A61K 47/10; A61K 47/34; A61K 47/38; A61K 47/42; A61P 17/02

(52) U.S. Cl. CPC A61K 31/58; A61K 9/0014; A61K 9/06; A61K 31/167; A61K 31/245; A61K 31/381; A61K 31/435; A61K 31/451; A61K 31/4706; A61K 31/5375; A61K 31/575; A61K 47/08; A61K 47/10; A61K 47/34; A61K 47/38; A61K 47/42; A61P 17/02

(57) ABSTRACT

Compositions and methods of treatment involving transpore delivery of an active ingredient are provided.

### CROSS-REFERENCE TO RELATED APPLICATION

This application is a continuation of U.S. application Ser. No. 18/336,163, filed Jun. 16, 2023, which is a continuation of U.S. application Ser. No. 18/046,350 (now abandoned), filed Oct. 13, 2022, which is a continuation of U.S. application Ser. No. 16/586,406, filed Sep. 27, 2019 (now abandoned), which claims priority benefit to U.S. Provisional Appl. No. 62/738,736, filed Sep. 28, 2018, the content of which are hereby incorporated by reference in their entireties.

### FIELD OF THE INVENTION

This invention is related to novel compositions for transpore delivery of a pharmaceutically active ingredient to a patient in need thereof. This invention also provides novel methods for the prevention or reduction of scars, as well as improving the size and appearance of scar tissue. In addition, this invention provides novel pharmaceutical compositions for the treatment of other skin conditions, such as inflammatory diseases (e.g., psoriasis, eczema), and for the management of discomfort or pain.

### BACKGROUND OF THE INVENTION

Transdermal drug delivery can be attempted through various dosage forms, for example, patches, creams and ointments. Each dosage form has its limitations. Patches are difficult to apply to curved surfaces, cumbersome and uncomfortable. Additionally, patches cause pain while peeling off and have poor aesthetic appeal. Dermal patches also exhibit reliability problems, not sticking predictably in different climates and degrees of skin oiliness. This limits the efficacy of transdermal drug delivery via patches. It is well-established that amplification of transdermal bioavailability by occlusion alone is inadequate to treat many maladies.

Semisolid preparations, like creams and ointments, overcome some of these drawbacks but have other limitations. For example, creams and ointments do not ensure persistent contact with the skin surface, as they can be easily wiped off by clothes, so repeated applications are required in case of chronic diseases. Creams and ointments also leave a sticky and greasy feel after application, which may lead to poor patient compliance. Additionally, as with transdermal patches, it is well-established that bioavailability of the active drug is often inadequate for treatment when delivered by creams and ointments. In these situations, only injections have been effective. Injections however are painful, expensive, and poorly tolerated by patients, especially when there is a need for repeat injections over time. Therefore, there is a need for development of a method of transpore drug delivery, bypassing the skin surface without an injection, which permits painless, comfortable and invisible application and thereby improves drug delivery and patient compliance.

### BRIEF SUMMARY OF THE INVENTION

Methods for transpore delivery of a steroid, bypassing the stratum corneum of the skin, to a patient suffering from a skin condition are provided herein. In one embodiment, the method comprises applying a liquid composition comprising about 1% to about 10% by weight of a steroid to an area of affected skin surface of the patient, said composition, when applied to the skin surface of the patient, achieves one or more of the following: (a) has a thickness of about 0.1 μm to about 10 μm in solid form, (b) forms a solid or semi-solid film, and (c) provides a mean Tₘₐₓ of from about 0.5 hours to about 8 hours, wherein the composition seeps into skin pores in liquid form and creates a biomechanical integration with the interior or the inside surface of said skin pores in solid form, and wherein said skin condition is selected from the group consisting of inflammatory skin conditions, hypertrophic scars, keloid scars, or a combination thereof.

Methods of stimulating procollagenase or collagenase production in a patient suffering from a skin condition are also provided herein. In one embodiment, the method comprises applying a liquid composition comprising about 1% to about 10% by weight of a steroid to an area of affected skin surface of the patient, said composition, when applied to the skin surface of the patient, achieves one or more of the following: (a) has a thickness of about 0.1 μm to about 10 μm in solid form, (b) forms a solid or semi-solid film, and (c) provides a mean Tₘₐₓ of from about 0.5 hours to about 8 hours, wherein the composition seeps into skin pores in liquid form and creates a biomechanical integration with the interior or the inside surface of said skin pores in solid form, and wherein said skin condition is hypertrophic scars or keloid scars, or a combination thereof.

Methods of stimulating collagenase activity in a patient suffering from a skin condition are also provided herein. In some embodiments, the method comprises applying a liquid composition comprising about 1% to about 10% by weight of a steroid to an area of affected skin surface of the patient, said composition, when applied to the skin surface of the patient, achieves one or more of the following: (a) has a thickness of about 0.1 μm to about 10 μm in solid form, (b) forms a solid or semi-solid film, and (c) provides a mean Tₘₐₓ of from about 0.5 hours to about 8 hours, wherein the composition seeps into skin pores in liquid form and creates a biomechanical integration with the interior or the inside surface of said skin pores in solid form, and wherein said skin condition is hypertrophic scars or keloid scars, or a combination thereof.

In certain embodiments of the methods, applying the liquid composition comprising about 1% to about 10% by weight of a steroid to an area of affected skin surface of the patient results in less than 10% of the steroid entering the systemic circulation of the patient after 8 hours of contact on the skin surface.

In certain embodiments, the procollagenase or collagenase production is stimulated by about 150% to about 500% after 48 hours.

In some embodiments of the methods, said area of affected skin surface is from about 1 cm² to about 500 cm².

In some embodiments of the methods, the composition provides a mean Cₘₐₓ of the steroid from about 10 pg/mL to about 1000 pg/mL when applied to the affected skin surface of the patient. In certain embodiments of the methods, the composition provides a mean Cₘₐₓ of from about 10 pg/mL to about 500 pg/mL when applied to the affected skin surface of the patient. In certain embodiments of the methods, the composition provides a mean Cₘₐₓ of the steroid from about 10 pg/mL to about 100 pg/mL when applied to the affected skin surface of the patient.

In some embodiments of the methods, the composition provides a mean flux of steroid from about 1 μg/cm²/hr to about 20 μg/cm²/hr when applied to the affected skin surface of the patient. In certain embodiments of the methods, the composition provides a mean flux of steroid from about 1 μg/cm²/hr to about 10 μg/cm²/hr when applied to the affected skin surface of the patient.

In some embodiments of the methods, the composition further comprises about 0% to about 9% by weight of silicone gel. In certain embodiments of the methods, the composition further comprises about 50% to about 99% by weight of pyroxylin, ether and alcohol.

In some embodiments of the methods, the composition further comprises about 0% to about 0.4% by weight of vitamin E. In some embodiments of the methods, the composition further comprises about 0.1% to about 0.4%, about 0.1% to about 0.3%, about 0.1% to about 0.2%, or about 0.1% by weight of vitamin E. In certain embodiments of the methods, the composition does not contain any vitamin E. In some embodiments, the composition comprises about 0% to about 9% by weight of silicone gel and no vitamin E.

In certain embodiments of the methods, the skin condition is an inflammatory skin condition. In certain embodiments of the methods, the skin condition is hypertrophic scars. In certain embodiments of the methods, the skin condition is keloid scars.

In some embodiments of the methods, the steroid is selected from the group consisting of one or more of clobetasol propionate, flurandrenolide, betamethasone dipropionate, diflorasone diacetate, desoximetasone, halobetasol propionate, fluocinonide, mometasone furoate, mometasone, halcinonide, desoximetasone, fluticasone propionate, triamcinolone acetonide, hydrocortisone valerate, fluocinolone acetonide, prednicarbate, desonide, hydrocortisone, fluocinolone acetonide, hydrocortisone valerate, alclometasone dipropionate, and other pharmaceutically acceptable salts thereof. In certain embodiments, the steroid is mometasone or a pharmaceutically acceptable salt thereof.

In some embodiments of the methods, about 0.5 mg to about 10 mg of said steroid is applied on to the affected skin surface in a daily dose. In some embodiments of the methods, about 0.05 ml to about 5 ml of said composition is applied on to the skin surface in a daily dose. In some embodiments, the solid or semi-solid film is kept on the skin surface for 2 to 7 days, for 1 to 3 weeks, or for 3 to 6 months, and is reapplied as needed. In some embodiments of the methods, the composition is applied on to the affected skin surface from 1 to 7 times a week.

In some embodiments of the methods, the solid or semi-solid film is an occlusive film.

Liquid compositions that dry to a solid or semi-solid film are also provided herein. In some embodiments, the liquid compositions comprise pyroxylin, ether, alcohol, and about 1% to about 10% by weight of steroid, said composition, when applied to an area of affected skin surface of a patient suffering from a skin condition, achieves one or more of the following: (a) has a thickness of about 0.1 μm to about 10 μm in solid form, (b) forms a solid or semi-solid film, and (c) provides a mean Tₘₐₓ of from about 0.5 hours to about 8 hours.

Liquid compositions that dry to a solid or semi-solid film comprising pyroxylin, ether, alcohol, about 1% to about 10% by weight of steroid, and about 0% to about 9% by weight of silicone gel, said composition, when applied to an area of affected skin surface of a patient suffering from a skin condition, achieves one or more of the following: (a) has a thickness of about 0.1 μm to about 10 μm in solid form, (b) forms a solid or semi-solid film, and (c) provides a mean Tₘₐₓ of from about 0.5 hours to about 8 hours.

In some embodiments, the solid or semi-solid film is an occlusive film.

In some embodiments of the liquid composition, the steroid is mometasone or a pharmaceutically acceptable salt thereof.

In some embodiments of the liquid composition, the liquid composition seeps into skin pores in liquid form and creates a biomechanical integration with the interior or the inside surface of said skin pores in solid form.

In some embodiments of the liquid composition, the composition provides a mean Cₘₐₓ of the steroid from about 10 pg/mL to about 1000 pg/mL when administered to the patient. In certain embodiments of the liquid composition, the composition provides a mean Cₘₐₓ of the steroid from about 10 pg/mL to about 500 pg/mL when administered to the patient. In certain embodiments of the liquid composition, the composition provides a mean Cₘₐₓ of the steroid from about 10 pg/mL to about 100 pg/mL when administered to the patient.

In some embodiments of the liquid composition, the composition provides a mean flux of the steroid from about 1 to about 20 μg/cm²/hr when applied to the affected skin surface of the patient.

Methods of treating a patient suffering from a skin condition are provided herein. In some embodiments, the method comprises applying the liquid composition containing the steroid to an area of affected skin surface of the patient, wherein the composition seeps into skin pores in liquid form and creates a biomechanical integration with the interior or the inside surface of said skin pores in solid form, and wherein said skin condition is selected from the group consisting of inflammatory skin conditions, hypertrophic scars, and keloid scars, or a combination thereof.

In certain embodiments, the skin condition is an inflammatory skin condition. In certain embodiments, the skin condition is hypertrophic scars. In certain embodiments, the skin condition is keloid scars. In certain embodiments, about 0.5 mg to about 10 mg of said steroid is applied on to the affected skin surface in a daily dose. In certain embodiments, about 0.1 ml to about 5 ml of said composition is applied on to the skin surface in a daily dose. In certain embodiments, the solid or semi-solid film is kept on the skin surface for 2 to 7 days, for 1 to 3 weeks, or for 3 to 6 months, and is reapplied as needed. In certain embodiments, the composition is applied on to the affected skin surface from 1 to 7 times a week. In some embodiments, the solid or semi-solid film is an occlusive film.

Liquid compositions that dry to a solid or semi-solid film for treating a patient suffering from a skin condition comprising about 0.001% to about 10% by weight of a biologic drug, said composition, when applied to an area of affected skin surface of the patient, forms a solid or semi-solid film, and said composition contains a pharmaceutically acceptable excipient selected from the group consisting of a polypeptide, a synthetic polymer, a surfactant, a liposome, a transfersome, an ethosome, a niosome, a solid lipid nanoparticle, or a combination thereof, wherein said skin condition is an inflammatory skin condition are provided herein.

In certain embodiments, the biologic drug is selected from the group consisting of one or more of certolizumab pegol, etanercept, adalimumab, infliximab, golimumab, ustekinumab, secukinumab, ixekizumab, brodalumab, abatacept, guselkumab, and tildrakizumab-asmn.

In some embodiments, the solid or semi-solid film is an occlusive film. In certain embodiments, the composition has a thickness of about 0.1 μm to about 10 μm in solid form. In certain embodiments, the composition seeps into skin pores in liquid form and creates a biomechanical integration with the interior or the inside surface of said skin pores in solid form. In certain embodiments, the biologic drug is delivered through skin pores, bypassing the stratum corneum of the skin, and interferes with the immune system. In certain embodiments, the inflammatory skin condition is acne. In certain embodiments, the inflammatory skin condition is skin cancer. In certain embodiments, the composition provides a mean flux of the biologic from about 0.5 μg/cm²/hr to about 20 μg/cm²/hr when applied to the affected skin surface of the patient. In certain embodiments, the composition provides a mean flux of the biologic drug from about 0.5 μg/cm²/hr to about 10 μg/cm²/hr when applied to the affected skin surface of the patient. In certain embodiments, the composition provides a mean flux of the biologic drug from about 0.5 μg/cm²/hr to about 5 μg/cm²/hr when applied to the affected skin surface of the patient.

In certain embodiments, the biologic drug is certolizumab pegol, and the composition, when administered to the patient, provides a mean Cₘₐₓ of from about 30 μg/mL to about 60 μg/mL and a mean Tₘₐₓ of from about 40 to about 200 hours. In certain embodiments, the biologic drug is etanercept, and the composition, when administered to the patient, provides a mean Cₘₐₓ of from about 0.5 μg/mL to about 4 μg/mL and a mean Tₘₐₓ of from about 30 to about 120 hours. In certain embodiments, the biologic drug is adalimumab, and the composition, when administered to the patient, provides a mean Cₘₐₓ of from about 2 μg/mL to about 8 μg/mL and a mean Tₘₐₓ of from about 60 to about 200 hours. In certain embodiments, the biologic drug is infliximab, and the composition, when administered to the patient, provides a mean Cₘₐₓ of from about 0.5 μg/mL to about 6 μg/mL and a mean terminal half-life of from about 7 to about 10 days. In certain embodiments, the biologic drug is golimumab, and the composition, when administered to the patient, provides a mean Cₘₐₓ of from about 1 μg/mL to about 4 μg/mL and a mean Tₘₐₓ of from about 1 to about 7 days. In certain embodiments, the biologic drug is ustekinumab, and the composition, when administered to the patient, provides a mean Cₘₐₓ of from about 80 μg/mL to about 180 μg/mL and a mean Tₘₐₓ of from about 6 to about 15 days. In certain embodiments, the biologic drug is secukinumab, and the composition, when administered to the patient, provides a mean Cₘₐₓ of from about 6 μg/mL to about 40 μg/mL and a mean Tₘₐₓ of from about 4 to about 8 days. In certain embodiments, the biologic drug is ixekizumab, and the composition, when administered to the patient, provides a mean Cₘₐₓ of from about 5 μg/mL to about 22 μg/mL and a mean Tₘₐₓ of from about 1 to about 5 days. In certain embodiments, the biologic drug is brodalumab, and the composition, when administered to the patient, provides a mean Cₘₐₓ of from about 8 μg/mL to about 24 μg/mL and a mean Tₘₐₓ of from about 2 to about 6 days. In certain embodiments, the biologic drug is abatacept, and the composition, when administered to the patient, provides a mean Cₘₐₓ of from about 150 μg/mL to about 500 g/mL and a mean terminal half-life of from about 5 to about 30 days. In certain embodiments, the biologic drug is guselkumab, and the composition, when administered to the patient, provides a mean Cₘₐₓ of from about 4 μg/mL to about 14 μg/mL and a mean Tₘₐₓ of from about 3 to about 8 days. In certain embodiments, the biologic drug is tildrakizumab-asmn, and the composition, when administered to the patient, provides a mean Cₘₐₓ of from about 4 μg/mL to about 12 μg/mL and a mean Tₘₐₓ of from about 4 to about 8 days.

Methods of treating a patient suffering from a skin condition, the method comprising applying the liquid composition comprising a biological drug to an area of affected skin surface of the patient, wherein the composition seeps into skin pores in liquid form and creates a biomechanical integration with the interior or the inside surface of said skin pores in solid form and wherein said skin condition is an inflammatory skin disorder are provided herein. In certain embodiments, about 0.05 mg to about 20 mg of a biologic drug is applied on to the affected skin surface in a daily dose. In certain embodiments, about 0.05 ml to about 5 ml of said composition is applied on to the affected skin surface in a daily dose. In certain embodiments, the solid or semi-solid film is kept on the skin surface for 2 to 7 days, or for 1 to 3 weeks, and is reapplied as needed. In certain embodiments, the composition is applied on to the affected skin surface from 1 to 7 times a week. In certain embodiments, the composition allows transpore delivery of the biologic drug bypassing the stratum corneum of the skin to said patient. In some embodiments, the solid or semi-solid film is an occlusive film.

Liquid compositions that dry to a solid or semi-solid film for treating a patient suffering from a skin condition, said liquid composition consisting essentially of about 5% to about 15% by weight of silicone gel, and no vitamin E are provided herein. The liquid composition does not contain any active ingredient. In certain embodiments, the liquid composition has a thickness of about 0.1 μm to about 10 μm in solid form. In certain embodiments, the composition seeps into skin pores in liquid form and creates a biomechanical integration with the interior or the inside surface of said skin pores in solid form.

Liquid compositions that dry to a solid or semi-solid film comprising about 0.1% to about 15% by weight of an anesthetic are provided herein. In some embodiments, the liquid composition, when applied to the skin surface of a patient, forms a solid or semi-solid film. In certain embodiments, the composition has a thickness of about 0.1 μm to about 10 μm in solid form. In certain embodiments, the composition seeps into skin pores in liquid form and creates a biomechanical integration with the interior or the inside surface of said skin pores in solid form. In some embodiments, the solid or semi-solid film is an occlusive film.

In some embodiments, the anesthetic included in the liquid composition is selected from the group consisting of one or more of articaine, benzocaine, bupivacaine, butamben, chloroprocaine, cocaine, cyclomethycaine, dibucaine, dimethocaine, etidocaine, levobupivacaine, lidocaine, mepivacaine, novocaine, oxybuprocaine, pramoxine, piperocaine, prilocaine, proparacaine, propoxycaine, proxymetacaine, ropivacaine, tetracaine, and trimecaine. In certain embodiments, the anesthetic is novocaine.

In some embodiments, the composition provides a mean Cₘₐₓ of the anesthetic from about 1 ng/mL to about 200 ng/ml when applied to the skin surface of the patient. In certain embodiments, the composition provides a mean Cₘₐₓ of the anesthetic from about 1 ng/ml to about 100 ng/ml when applied to the skin surface of the patient.

In some embodiments, the composition provides a mean flux of the anesthetic from about 1 μg/cm²/hr to about 20 μg/cm²/hr when applied to the skin surface of the patient. In some embodiments, the composition provides a mean flux of the anesthetic from about 1 μg/cm²/hr to about 10 μg/cm²/hr when applied to the skin surface of the patient.

In some embodiments, the composition provides a mean time for onset of action of the anesthetic from about 1 minute to about 2 hours when applied to the skin surface of the patient. In certain embodiments, the composition provides a mean time for onset of action of the anesthetic from about 1 minute to about 15 minutes when applied to the skin surface of the patient.

In some embodiments, the liquid composition comprising the anesthetic is applied to the skin surface of the patient. In certain embodiments, the area of skin surface is from about 1 cm² to about 500 cm². In certain embodiments, about 5 mg to about 1000 mg of said anesthetic is applied on to the skin surface in a single dose or in multiple doses. In certain embodiments, about 0.05 ml to about 5 ml of said composition is applied on to the skin surface in a single dose or in multiple doses. In some embodiments, the composition is applied on to the skin surface from 10 minutes to 3 hours prior to a medical procedure. In some embodiments, the procedure is injection, vaccination, biopsy, endoscopy, acupuncture, mole removal, general surgery, or a medical procedure that causes pain or discomfort to the patient.

Liquid compositions that dry to a solid or semi-solid film comprising about 0.001% to about 15% of an active ingredient by weight, about 0% to about 9% of silicone gel by weight, pyroxylin, ether, and alcohol, wherein said composition, when applied to the skin surface of a patient, forms a solid or semi-solid film, are provided herein. In certain embodiments, the liquid composition comprises 0% of silicone gel by weight. In certain embodiments, the thickness of the composition is about 0.1 μm to about 10 μm in solid form. In certain embodiments, the composition seeps into skin pores in liquid form and creates a biomechanical integration with the interior or the inside surface of said skin pores in solid form. In certain embodiments the active ingredient is a steroid, a biologic drug, or an anesthetic. In some embodiments, the composition provides a mean Cₘₐₓ of the active ingredient from about 10 pg/mL to about 500 μg/mL when applied to the skin surface of the patient. In some embodiments, the composition provides a mean flux of the active ingredient from about 1 μg/cm²/hr to about 20 μg/cm²/hr when applied to the skin surface of the patient. In some embodiments, the composition is applied to the skin surface of the patient, wherein the area of skin surface is from about 1 cm² to about 500 cm². In some embodiments, about 0.05 mg to about 1000 mg of said active ingredient is applied on to the skin surface in a daily dose. In some embodiments, about 0.05 ml to about 5 ml of said composition is applied on to the skin surface in a daily dose. In some embodiments, the solid or semi-solid film is kept on the skin surface for 2 to 7 days, for 1 to 3 week, for 3 to 6 months, and is reapplied as needed. In some embodiments, the composition is applied on to the skin surface from 1 to 7 times a week.

Liquid compositions that dry to a solid or semi-solid film comprising pyroxylin, ether, alcohol, and about 0.001% to about 10% of a biologic drug by weight, said composition, when applied to an area of affected skin surface of a patient suffering from a skin condition, achieves one or more of the following: (a) has a thickness of about 0.1 μm to about 10 μm in solid form, and (b) forms a solid or semi-solid film are provided herein. In some embodiments, the solid or semi-solid film is an occlusive film.

Liquid compositions that dry to a solid or semi-solid film comprising pyroxylin, ether, alcohol, about 0.001% to about 10% of a biologic drug by weight, and about 0% to about 9% by weight of silicone gel, said composition, when applied to an area of affected skin surface of a patient suffering from a skin condition, achieves one or more of the following: (a) has a thickness of about 0.1 μm to about 10 μm in solid form, and (b) forms a solid or semi-solid film are provided herein. In some embodiments, the solid or semi-solid film is an occlusive film.

Liquid compositions that dry to a solid or semi-solid film comprising pyroxylin, ether, alcohol, and about 0.1% to about 15% of an anesthetic by weight, said composition, when applied to a patient in need of pain management prior to a medical procedure, achieves one or more of the following: (a) has a thickness of about 0.1 μm to about 10 μm in solid form, (b) forms a solid or semi-solid film, and (c) provides a mean time for onset of action of from about 1 minute to about 2 hours are provided herein. In some embodiments, the solid or semi-solid film is an occlusive film.

### BRIEF DESCRIPTION OF THE DRAWINGS

FIG. 1A provides a sectional view of the structure of skin pores.

FIG. 1B provides a sectional view of the transpore delivery of liquid drug compositions into the skin pores.

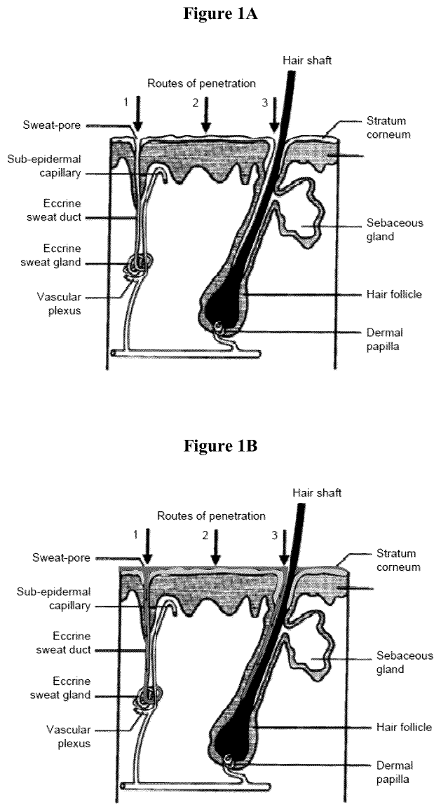

### DETAILED DESCRIPTION OF THE INVENTION

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “steroid” as used herein refers to the free base or a pharmaceutically acceptable salt form of a steroid.

The term “free base” as used herein includes, but is not limited to, the unprotonated form of a therapeutic agent, molecule, or compound. Additionally, “free base” includes, but is not limited to, the neutral form of a molecule or compound.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salt” as used herein includes those salts which are within the scope of sound judgment. For example, if the compound is cationic, or has a functional group which may be cationic (e.g. —NH₂ may be —NH₃⁺), then an acid addition salt may be formed with a suitable anion. Examples of suitable inorganic anions include, but are not limited to, those derived from the following inorganic acids: hydrochloric acid, nitric acid, nitrous acid, phosphoric acid, sulfuric acid, sulphurous acid, hydrobromic acid, hydroiodic acid, hydrofluoric acid, phosphoric acid and phosphorous acids. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfonate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

If the compound is anionic, or has a functional group which may be anionic (e.g. —COOH may be —COO⁻), then a base salt may be formed with a suitable cation. Examples of suitable inorganic cations include, but are not limited to, metal cations, such as an alkali or alkaline earth metal cation, ammonium and substituted ammonium cations, as well as amines. Examples of suitable metal cations include sodium (Na⁺) potassium (K⁺), magnesium (Mg²⁺), calcium (Ca²⁺), zinc (Zn²⁺), and aluminum (Al³⁺). Examples of suitable organic cations include, but are not limited to, ammonium ion (i.e. NH₄⁺) and substituted ammonium ions (e.g. NH₃R⁺, NH₂R₂⁺, NHR₃⁺, NH₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺. Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2011).

The term “transpore delivery” refers to the delivery of an agent into and/or through the skin pores for local or topical therapy.

The term “Cₘₐₓ” as used herein refers to the maximum plasma concentration of a drug after it is administered to a patient.

The term “Tₘₐₓ” as used herein refers to the time required to reach the maximal plasma concentration (“Cₘₐₓ”) after administration of a drug.

The term “terminal half-life” (“t_{1/2}”) as used herein refers to the time it takes for the blood plasma concentration of a biologic drug to halve its steady-state.

The term “treating” or “treatment” as used herein refers to the administration of a composition to a subject for therapeutic purposes.

The term “administration” or “administering” as used herein refers to the act of a physician or other medical professional prescribing a pharmaceutical composition of the invention for a subject.

The term “mean” refers to an average value in a patient population. For example, a “mean Cₘₐₓ” refers to an average of the maximum plasma concentrations of a drug in a patient population.

The term “flux” refers to the rate at which the pharmaceutically active ingredient crosses the skin barrier.

The term “affected skin surface” refers to an area of the skin that demonstrates the symptoms of a skin disease.

The term “occlusive film” refers to a solid or semi-solid film that is an impermeable thin layer of material that covers the skin.

### Pharmaceutical Compositions

The present disclosure provides a liquid composition that dries to a solid or semi-solid film. When applied to the skin, the liquid composition covers the surface of the skin and seeps into the skin pores. When the liquid dries, the solvent evaporates and the resultant film picks up local moisture and swells, creating a film with reliable biomechanical integration with the interior microarchitecture of the skin on the inside surface of the skin pores. This film is tangible, yet barely visible, avoiding compliance issues and providing significantly enhanced reliability.

In one embodiment, the liquid composition once dried, can also be described as an intrapore drug-eluting stent or stent-like structure.

There are two types of skin pores: pilosebaceous follicles or eccrine sweat glands. A pilosebaceous follicle has a diameter of approximately 40-80 μm, and an eccrine sweat gland has a diameter of approximately 5-10 μm. The liquid compositions of the present disclosure seep into skin pores in liquid form and create a biomechanical integration with the interior or the inside surface of said skin pores in solid form.

In some embodiments, the solid or semi-solid film formed by the liquid composition is an occlusive film. In some embodiments, the thickness of the solid or semi-solid film that forms when the liquid composition dries ranges from about 0.1 to about 10 μm, from about 0.1 to about 5 μm, from about 0.1 to about 2 μm, from about 0.5 to about 10 μm, from about 0.5 to about 5 μm, from about 0.5 to about 2 μm, from about 1 to about 10 μm, from about 1 to about 5 μm, from about 1 to about 2 μm, from about 3 to about 10 μm, from about 3 to about 5 μm, about 5 to about 10 μm, or from about 7 to about 10 μm. In some embodiments, the thickness of the film ranges from about 3 to about 4 μm.

In some embodiments, the liquid composition comprises one or more film forming polymers. In certain embodiments, the total weight percentage of the one or more film forming polymers is from about 1% to about 10%, from about 3% to about 10%, from about 5% to about 10%, from about 7% to about 10%, from about 1% to about 8%, from about 3% to about 8%, from about 5% to about 8%, from about 7% to about 8%, from about 1% to about 6%, from about 3% to about 6%, from about 5% to about 6%, from about 1% to about 4%, from about 2% to about 4%, or from about 1% to about 2%. In some embodiments, the total weight percentage of the one or more film forming polymers is about 1%, about 2%, about 3%, about 4%, about 5%, about 6%, about 7%, about 8%, about 9%, or about 10%.

Examples of suitable film-forming polymers for the liquid composition include, but are not limited to, nitrocellulose, cellulose esters, cellulose ethers, cellulose esters-ethers, cellulose acylate, polyquaternium hyaluronic acid, or any combinations thereof. In some embodiments, the film-forming polymer is nitrocellulose. In one embodiment, the nitrocellulose film-forming polymer is pyroxylin.

In some embodiments, the liquid composition comprises nitrocellulose, ether and alcohol. In certain embodiments, the total weight percentage of nitrocellulose, ether and alcohol is from about 50% to about 99%, from about 60% to about 99%, from about 70% to about 99%, from about 80% to about 99%, from about 90% to about 99%, from about 50% to about 90%, from about 60% to about 90%, from about 70% to about 90%, from about 80% to about 90%, from about 50% to about 80%, from about 60% to about 80%, from about 70% to about 80%, from about 50% to about 70%, or from about 60% to about 70%.

In some embodiments, the liquid composition comprises pyroxylin, ether and alcohol. In certain embodiments, the total weight percentage of pyroxylin, ether and alcohol is from about 50% to about 99%, from about 60% to about 99%, from about 70% to about 99%, from about 80% to about 99%, from about 90% to about 99%, from about 50% to about 90%, from about 60% to about 90%, from about 70% to about 90%, from about 80% to about 90%, from about 50% to about 80%, from about 60% to about 80%, from about 70% to about 80%, from about 50% to about 70%, or from about 60% to about 70%.

In some embodiments, the composition comprises about 1% to about 10% of pyroxylin by weight. In some embodiments, the composition comprises about 1%, about 2%, about 3%, about 4%, about 5%, about 6%, about 7%, about 8%, about 9%, about 10% of pyroxylin by weight. In some embodiments, the composition comprises about 40% to about 75% of ether by weight. In some embodiments, the composition comprises about 40% to about 50%, about 40% to about 60%, about 50% to about 60%, about 50% to about 75%, or about 60% to about 75% of ether by weight. In some embodiments, the composition comprises about 20% to about 30% of alcohol. In some embodiments, the composition comprises about 20%, about 21%, about 22%, about 23%, about 24%, about 25%, about 26%, about 27%, about 28%, about 29%, or about 30% of alcohol by weight. Examples of ethers include, but are not limited to, diethyl ether and polyoxyethylene lauryl ether. Examples of alcohol include, but are not limited to, ethanol and isopropanol. In some embodiments, the proportion of the weight of alcohol to the weight of ether is about 1:4, about 1:3.5, about 1:3, about 1:2.5, or about 1:2. In one embodiment, the liquid composition comprises 4 g nitrocellulose in 100 mL of a mixture of 25 mL alcohol and 75 mL ether.

In yet other embodiments, the liquid composition comprises one or more plasticizers. In certain embodiments, the total weight percentage of the one or more plasticizers is from about 1% to about 20%, from about 5% to about 20%, from about 10% to about 20%, from about 15% to about 20%, from about 1% to about 16%, from about 5% to about 16%, from about 10% to about 16%, from about 1% to about 12%, from about 5% to about 12%, from about 8% to about 12%, from about 1% to about 8%, or from about 4% to about 8%. In some embodiments, the total weight percentage of the plasticizer is about 1%, about 2%, about 3%, about 4%, about %, about 6%, about 7%, about 8%, about 9%, about 10%, about 11%, about 12%, about 13%, about 14%, about 15%, about 16%, about 17%, about 18%, about 19%, or about 20%.

Examples of suitable plasticizers for the liquid composition include, but are not limited to, polyethylene glycol, propylene glycol, polyesters (e.g. poly (lactic acid) and poly(lactide-co-glycolide)), polyesteramides, diesters/triesters of acids, diesters/triesters of alcohols, and combinations thereof.

In some embodiments, the liquid composition further comprises about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, about 0.1% to 1% by weight of silicone gel. In one embodiment, the liquid composition comprises no silicone gels. Suitable silicone gels contain the recurring group —SiR₂O— wherein R is a radical such as an alkyl, phenyl, or vinyl group which may be substituted or unsubstituted. An example of a silicone gel suitable for the liquid composition is marketed under the tradename SILASTIC® manufactured by Dow Corning. Other suitable silicon gels include, but are not limited to, phenyl trimethicone (e.g., Dow Corning 556) or a non-volatile polydimethylsiloxane.

In some embodiments, the liquid composition further comprises about 0.1% to about 0.4%, about 0.1% to about 0.3%, about 0.1% to about 0.2% by weight of vitamin E. In one embodiment, the liquid composition comprises no vitamin E.

In some embodiments, the liquid composition further comprises a pharmaceutically acceptable excipient. Examples of the pharmaceutically acceptable excipients include, but are not limited to: polypeptides, synthetic polymers, surfactants, liposomes, transfersomes, ethosomes, niosomes, solid lipid nanoparticles, chemical penetrating enhancers, or a combination thereof.

In some embodiments, the liquid composition comprises about 0.1% to about 20% by weight of a pharmaceutically acceptable excipient. In some embodiments, the liquid composition comprises about 1% to about 20%, about 3% to about 20%, about 5% to about 20%, about 8% to about 20%, about 10% to about 20%, about 12% to about 20%, about 15% to about 20%, about 18% to about 20%, about 0.1% to about 15%, about 1% to about 15%, about 3% to about 15%, about 5% to about 15%, about 8% to about 15%, about 10% to about 15%, about 12% to about 15%, about 0.1% to about 12%, about 1% to about 12%, about 3% to about 12%, about 5% to about 12%, about 8% to about 12%, about 10% to about 12%, about 8% to about 10%, about 0.1% to about 8%, about 1% to about 8%, about 3% to about 8%, about 5% to about 8%, about 0.1% to about 3%, about 1% to about 3%, or about 0.1% to about 1% by weight of a pharmaceutically acceptable excipient.

In some embodiments, the liquid composition comprises surfactants. Examples of surfactants include, but are not limited to, alkylglucosides, alkylmaltosides, alkylthioglucosides, lauryl macrogolglycerides, fatty acids, lower alcohol fatty acid esters, polyoxyethylene alkylphenols, polyethylene glycol fatty acids esters, polypropylene glycol fatty acid esters, glycerol fatty acid esters, acetylated, glycerol fatty acid esters, polyethylene glycol glycerol fatty acid esters, polyglyceryl fatty acid esters, polyoxyethylene sorbitan fatty acid esters, polyoxyethylene glycerides, polyoxyethylene sterols, polyoxyethylene vegetable oils, polyoxyethylene hydrogenated vegetable oils, reaction mixtures of polyols and at least one member of the group consisting of fatty acids, vegetable oils, hydrogenated vegetable oils, and sterols, sugar esters, sugar ethers, sucroglycerides, fatty acid salts, bile salts, phospholipids, phosphoric acid esters, carboxylates, sulfates, sulfonates, or a combination thereof.

In some embodiments, the liquid composition comprises one or more of liposomes, transfersomes, ethosomes, niosomes, or a combination thereof. The types of lipids and amphiphiles that can act as liposomes, transfersomes, ethosomes or niosomes in the liquid composition include, but are not limited to, phosphotidylcholine, phosphotidylserine, phosphatidylethanolamine, phosphatidylinositol, 1,2-dilauroyl-sn-glycero-3-phosphocholine, 1,2-dioleoyl-sn-glycero-3-[phospho-L-serine] and the salt thereof, dipalmitoylphosphatidylcholine, distearoylphosphatidylcholine, dipalmitoylphosphotidylseine, dipalmitoylphosphatidylglycerol, 1,2-dilauroyl-sn-glycero-3-phosphocholine, 1-stearoyl-2-linoleoyl-sn-glycero-3-[phospho-L-serine] and the salt thereof, dioleoylphosphatidylcholine, sphingomyelin, gangliosides, cholesterol, lipids conjugated to diene, methacrylate and thiol groups, diotadecyldimethyl ammonium bromide, diotadecyldimethyl ammonium chloride, and dioleoyl trimethylammonium propane, sucrose ester surfactants, polyoxyethylene alkyl ether surfactants, or a combination thereof.

In some embodiments, the liquid composition comprises chemical penetration enhancers. Chemical penetration enhancers interact with the lipid domain of the stratum corneum, disrupting these, and causing fluidization. Examples of chemical penetration enhancers include, but are not limited to, dimethylsulphoxide, azone, pyrrolidones, fatty acids, fatty alcohols, peptides, trypsin, or a combination thereof.

In some embodiments, the liquid composition that dries to a solid or semi-solid film comprises one or more active ingredients. In certain embodiments, the liquid composition comprises about 0.001% to about 10%, about 0.01% to about 10%, about 0.1% to about 10%, about 0.5% to about 10%, about 1% to about 10%, about 3% to about 10%, about 5% to about 10%, about 7% to about 10%, about 9% to about 10%, about 0.001% to about 8%, about 0.01% to about 8%, about 0.1% to about 8%, about 0.5% to about 8%, about 1% to about 8%, about 3% to about 8%, about 5% to about 8%, about 7% to about 8%, about 0.001% to about 6%, about 0.01% to about 6%, about 0.1% to about 6%, about 0.5% to about 6%, about 1% to about 6%, about 3% to about 6%, about 5% to about 6%, about 0.001% to about 4%, about 0.01% to about 4%, about 0.1% to about 4%, about 0.5% to about 4%, about 1% to about 4%, about 3% to about 4%, about 0.001% to about 2%, about 0.01% to about 2%, about 0.1% to about 2%, about 0.5% to about 2%, about 1% to about 2%, about 0.001% to about 1%, about 0.01% to about 1%, about 0.1% to about 1%, or about 0.5% to about 1% by weight of the active ingredient.

In some embodiments, the liquid composition results in about 0% to about 10%, of the active ingredient entering the systemic circulation of the patient after 8 hours of contact on the skin surface. In certain embodiments, the steroid composition results in about 0% to about 8%, about 0% to about 6%, about 0% to about 4%, about 0% to about 2%, about 2% to about 10%, about 2% to about 8%, about 2% to about 6%, about 2% to about 4%, about 4% to about 10%, about 4% to about 8%, about 4% to about 6%, about 6% to about 10%, about 6% to about 8%, or about 8% to about 10% of the active ingredient entering the systemic circulation of the patient after 8 hours of contact on the surface. In certain embodiments, the liquid composition results in about 0%, about 0.1%, about 0.2%, about 0.3%, about 0.4%, about 0.5%, about 1%, about 2%, about 3%, about 4%, about 5%, about 6%, about 7%, about 8%, about 9%, or about 10% of the active ingredient entering the systemic circulation of the patient after 8 hours of contact on the surface.

In some embodiments, the liquid composition that dries to a solid or semi-solid film comprises one or more steroids (“the steroid composition”). In some embodiments, the steroid composition comprises about 0.5% to about 10%, about 1% to about 10%, about 3% to about 10%, about 5% to about 10%, about 7% to about 10%, about 9% to about 10%, about 0.5% to about 8%, about 1% to about 8%, about 3% to about 8%, about 5% to about 8%, about 7% to about 8%, about 0.5% to about 6%, about 1% to about 6%, about 3% to about 6%, about 5% to about 6%, about 0.5% to about 4%, about 1% to about 4%, about 3% to about 4%, about 0.5% to about 2%, or about 1% to about 2% by weight of steroids. In certain embodiments, the steroid composition comprises about 1% to about 10% by weight of steroids.

In some embodiments, the steroid composition comprises one steroid. In other embodiments, the steroid composition comprises a mixture of two or more steroids. In some embodiments, the steroid is a corticosteroid. In some embodiments, the steroid is selected from the group consisting of one or more of clobetasol propionate, flurandrenolide, betamethasone dipropionate, diflorasone diacetate, desoximetasone, halobetasol propionate, fluocinonide, mometasone furoate, mometasone, halcinonide, desoximetasone, fluticasone propionate, triamcinolone acetonide, hydrocortisone valerate, fluocinolone acetonide, prednicarbate, desonide, hydrocortisone, fluocinolone acetonide, hydrocortisone valerate, alclometasone dipropionate, and other pharmaceutically acceptable salts thereof. In one embodiment, the steroid is mometasone or a pharmaceutically acceptable salt thereof.

In some embodiments, the steroid composition comprises about 0.5% to about 10%, about 1% to about 10%, about 3% to about 10%, about 5% to about 10%, about 7% to about 10%, about 9% to about 10%, about 0.5% to about 8%, about 1% to about 8%, about 3% to about 8%, about 5% to about 8%, about 7% to about 8%, about 0.5% to about 6%, about 1% to about 6%, about 3% to about 6%, about 5% to about 6%, about 0.5% to about 4%, about 1% to about 4%, about 3% to about 4%, about 0.5% to about 2%, or about 1% to about 2% by weight of mometasone. In certain embodiments, the steroid composition comprises about 1% to about 10% by weight of mometasone. The weight percentages of pharmaceutically acceptable salts of mometasone are adjusted based on the weight percentages of the free base.

In one embodiment, the steroid composition, when applied to the skin surface of the patient, provides a mean Cₘₐₓ of from about 10 pg/mL to about 1000 pg/mL. In certain embodiments, the steroid composition, when applied to the skin surface of the patient, provides a mean Cₘₐₓ of from about 10 pg/mL to about 800 pg/mL, from about 10 pg/mL to about 500 pg/mL, from about 10 pg/mL to about 300 pg/mL, from about 10 pg/mL to about 200 pg/mL, from about 10 pg/mL to about 100 pg/mL, from about 30 pg/mL to about 1000 pg/mL, from about 30 pg/mL to about 800 pg/mL, from about 30 pg/mL to about 500 pg/mL, from about 30 pg/mL to about 300 pg/mL, from about 30 pg/mL to about 200 pg/mL, from about 30 pg/mL to about 100 pg/mL, from about 50 pg/mL to about 1000 pg/mL, from about 50 pg/mL to about 800 pg/mL, from about 50 pg/mL to about 500 pg/mL, from about 50 pg/mL to about 300 pg/mL, from about 50 pg/mL to about 200 pg/mL, from about 50 pg/mL to about 100 pg/mL, from about 80 pg/mL to about 1000 pg/mL, from about 80 pg/mL to about 800 pg/mL, from about 80 pg/mL to about 500 pg/mL, from about 80 pg/mL to about 300 pg/mL, from about 80 pg/mL to about 200 pg/mL, from about 80 pg/mL to about 100 pg/mL, from about 100 pg/mL to about 1000 pg/mL, from about 100 pg/mL to about 800 pg/mL, from about 100 pg/mL to about 500 pg/mL, from about 100 pg/mL to about 300 pg/mL, from about 100 pg/mL to about 200 pg/mL, from about 200 pg/mL to about 1000 pg/mL, from about 200 pg/mL to about 800 pg/mL, from about 200 pg/mL to about 500 pg/mL, from about 200 pg/mL to about 300 pg/mL, from about 300 pg/mL to about 1000 pg/mL, from about 300 pg/mL to about 800 pg/mL, from about 300 pg/mL to about 500 pg/mL, from about 400 pg/mL to about 1000 pg/mL, from about 400 pg/mL to about 800 pg/mL, from about 400 pg/mL to about 500 pg/mL, from about 500 pg/mL to about 1000 pg/mL, from about 500 pg/mL to about 800 pg/mL, from about 600 pg/mL to about 1000 pg/mL, from about 600 pg/mL to about 800 pg/mL, from about 700 pg/mL to about 1000 pg/mL, from about 700 pg/mL to about 800 pg/mL, from about 800 pg/mL to about 1000 pg/mL, or from about 900 pg/mL to about 1000 pg/mL.

In some embodiments, the steroid composition, when applied to the skin surface of the patient, provides a mean Tₘₐₓ of from about 0.5 hours to about 10 hours. In certain embodiments, the steroid composition, when applied to the skin surface of the patient, provides a mean Tₘₐₓ of from about 0.5 hours to about 8 hours, from about 0.5 hours to about 5 hours, from about 0.5 hours to about 3 hours, from about 0.5 hours to about 2 hours, from about 1 hour to about 10 hours, from about 1 hour to about 8 hours, from about 1 hour to about 5 hours, from about 1 hour to about 3 hours, from about 1 hour to about 2 hours, from about 2 hour to about 10 hours, from about 2 hours to about 8 hours, from about 2 hours to about 5 hours, from about 2 hours to about 3 hours, from about 3 hours to about 10 hours, from about 3 hours to about 8 hours, from about 3 hours to about 5 hours, from about 4 hours to about 10 hours, from about 4 hours to about 8 hours, from about 4 hours to about 5 hours, from about 5 hours to about 10 hours, from about 5 hours to about 8 hours, from about 6 hours to about 10 hours, from about 6 hours to about 8 hours, from about 7 hours to about 10 hours, or from about 7 hours to about 8 hours.

In some embodiments, the steroid composition, when applied to the skin surface of the patient, provides a mean flux of from about 1 μg/cm²/hr to about 20 g/cm²/hr. In some embodiments, the steroid composition, when applied to the skin surface of the patient, provides a mean flux of from about 1 μg/cm²/hr to about 15 μg/cm²/hr, from about 1 μg/cm²/hr to about 10 μg/cm²/hr, from about 1 μg/cm²/hr to about 5 g/cm²/hr, from about 3 μg/cm²/hr to about 20 μg/cm²/hr, from about 3 μg/cm²/hr to about 15 μg/cm²/hr, from about 3 μg/cm²/hr to about 10 μg/cm²/hr, from about 3 μg/cm²/hr to about 5 μg/cm²/hr, from about 5 μg/cm²/hr to about 20 μg/cm²/hr, from about 5 μg/cm²/hr to about 15 μg/cm²/hr, from about 5 μg/cm²/hr to about 10 μg/cm²/hr, from about 8 μg/cm²/hr to about 20 μg/cm²/hr, from about 8 μg/cm²/hr to about 15 μg/cm²/hr, from about 8 μg/cm²/hr to about 10 μg/cm²/hr, from about 10 μg/cm²/hr to about 20 μg/cm²/hr, from about 10 μg/cm²/hr to about 15 μg/cm²/hr, from about 12 μg/cm²/hr to about 20 μg/cm²/hr, from about 12 μg/cm²/hr to about 15 μg/cm²/hr, from about 15 μg/cm²/hr to about 20 μg/cm²/hr, or from about 18 μg/cm²/hr to about 20 μg/cm²/hr.

In other embodiments, the liquid composition that dries to a solid or semi-solid film comprises one or more biologic (“the biologic composition”). Biologics are biological drugs that include a wide range of products such as vaccines, blood and blood components, allergenics, somatic cells, gene therapy, tissues, and proteins. Biologics can be composed of sugars, proteins, or nucleic acids or complex combinations of these substances, or may be living entities such as cells and tissues. Biologics can be isolated from a variety of sources-human, animal, or microorganism.

In some embodiments, the one or more biologic in the biologic composition is a recombinant therapeutic protein. In some embodiments, the biologic is one that is capable of interfering with the patient's immune system. Examples of a biologic that is capable of interfering with the patient's immune system include, but are not limited to, certolizumab pegol, etanercept, adalimumab, infliximab, golimumab, ustekinumab, secukinumab, ixekizumab, brodalumab, abatacept, guselkumab, tildrakizumab-asmn, or biosimilars thereof (e.g., infliximab-abda.)

In some embodiments, the biologic composition comprises about 0.001% to about 10%, about 0.01% to about 10%, about 0.1% to about 10%, about 0.5% to about 10%, about 1% to about 10%, about 3% to about 10%, about 5% to about 10%, about 7% to about 10%, about 0.001% to about 8%, about 0.01% to about 8%, about 0.1% to about 8%, about 0.5% to about 8%, about 1% to about 8%, about 3% to about 8%, about 5% to about 8%, about 7% to about 8%, about 0.001% to about 6%, about 0.01% to about 6%, about 0.1% to about 6%, about 0.5% to about 6%, about 1% to about 6%, about 3% to about 6%, about 5% to about 6%, about 0.001% to about 4%, about 0.01% to about 4%, about 0.1% to about 4%, about 0.5% to about 4%, about 1% to about 4%, about 3% to about 4%, about 0.001% to about 2%, about 0.01% to about 2%, about 0.1% to about 2%, about 0.5% to about 2%, about 1% to about 2%, about 0.001% to about 1%, about 0.01% to about 1%, about 0.1% to about 1%, or about 0.5% to about 1% by weight of one or more biologic. In some embodiments, the biologic composition comprises about 0.001% to about 10% by weight of one or more biologic.

In some embodiments, the biologic composition, when applied to the skin surface of the patient, provides a mean Cₘₐₓ of from about 0.5 μg/mL to about 600 μg/mL. In some embodiments, the biologic composition, when applied to the skin surface of the patient, provides a mean Cₘₐₓ of from about 0.5 μg/mL to about 10 μg/mL, from about 0.5 μg/mL to about 8 μg/mL, from about 0.5 μg/mL to about 6 μg/mL, from about 0.5 g/mL to about 4 μg/mL, from about 0.5 μg/mL to about 2 μg/mL, from about 1 μg/mL to about 20 μg/mL, from about 1 μg/mL to about 16 μg/mL, from about 1 μg/mL to about 12 μg/mL, from about 1 μg/mL to about 8 μg/mL, from about 1 μg/mL to about 4 μg/mL, from about 3 μg/mL to about 40 μg/mL, from about 3 μg/mL to about 35 μg/mL, from about 3 μg/mL to about 30 μg/mL, from about 3 μg/mL to about 25 μg/mL, from about 3 μg/mL to about 20 μg/mL, from about 3 μg/mL to about 15 μg/mL, from about 3 μg/mL to about 10 μg/mL, from about 5 μg/mL to about 60 μg/mL, from about 5 μg/mL to about 50 μg/mL, from about 5 μg/mL to about 40 μg/mL, from about 5 μg/mL to about 30 μg/mL, from about 5 μg/mL to about 20 μg/mL, from about 5 μg/mL to about 10 μg/mL, from about 3 μg/mL to about 10 μg/mL, from about 20 μg/mL to about 200 μg/mL, from about 20 μg/mL to about 160 μg/mL, from about 20 μg/mL to about 120 μg/mL, from about 20 μg/mL to about 80 μg/mL, from about 20 μg/mL to about 60 μg/mL, from about 20 μg/mL to about 40 g/mL, from about 60 μg/mL to about 300 μg/mL, from about 60 μg/mL to about 250 g/mL, from about 60 μg/mL to about 200 μg/mL, from about 60 μg/mL to about 150 μg/mL, from about 60 μg/mL to about 100 μg/mL, from about 100 μg/mL to about 600 μg/mL, from about 100 μg/mL to about 500 μg/mL, from about 100 μg/mL to about 400 μg/mL, from about 100 μg/mL to about 300 μg/mL, or from about 100 μg/mL to about 200 μg/mL. In one embodiment, the biologic composition provides a mean Cₘₐₓ of from about 30 μg/mL to about 60 μg/mL. In one embodiment, the biologic composition provides a mean Cₘₐₓ of from about 0.5 μg/mL to about 4 μg/mL. In one embodiment, the biologic composition provides a mean Cₘₐₓ of from about 2 μg/mL to about 8 μg/mL. In one embodiment, the biologic composition provides a mean Cₘₐₓ of from about 0.5 μg/mL to about 6 μg/mL. In one embodiment, the biologic composition provides a mean Cₘₐₓ of from about 1 μg/mL to about 4 μg/mL. In one embodiment, the biologic composition provides a mean Cₘₐₓ of from about 80 μg/mL to about 180 μg/mL. In one embodiment, the biologic composition provides a mean Cₘₐₓ of from about 6 μg/mL to about 40 μg/mL. In one embodiment, the biologic composition provides a mean Cₘₐₓ of from about 5 μg/mL to about 22 μg/mL. In one embodiment, the biologic composition provides a mean Cₘₐₓ of from about 8 μg/mL to about 24 μg/mL. In one embodiment, the biologic composition provides a mean Cₘₐₓ of from about 150 μg/mL to about 500 μg/mL. In one embodiment, the biologic composition provides a mean Cₘₐₓ of from about 4 μg/mL to about 14 μg/mL. In one embodiment, the biologic composition provides a mean Cₘₐₓ of from about 4 μg/mL to about 12 μg/mL.

In some embodiments, the biologic composition, when applied to the skin surface of the patient, provides a mean Tₘₐₓ of from about 20 hours to about 40 days. In some embodiments, the biologic composition, when applied to the skin surface of the patient, provides a mean Tₘₐₓ of from about 20 hours to about 300 hours, from about 20 hours to about 280 hours, from about 20 hours to about 260 hours, from about 20 hours to about 240 hours, from about 20 hours to about 220 hours, from about 20 hours to about 200 hours, from about 20 hours to about 180 hours, from about 20 hours to about 160 hours, from about 20 hours to about 140 hours, from about 20 hours to about 120 hours, from about 20 hours to about 100 hours, from about 20 hours to about 80 hours, from about 20 hours to about 60 hours, from about 20 hours to about 40 hours, from about 1 day to about 10 days, from about 1 day to about 8 days, from about 1 day to about 6 days, from about 1 day to about 4 days, from about 3 days to about 10 days, from about 3 days to about 8 days, from about 3 days to about 5 days, from about 5 days to about 40 days, from about 5 to about 30 days, from about 5 days to about 20 days, from about 5 days to about 10 days. In one embodiment, the biologic composition provides a mean Tₘₐₓ of from about 40 hours to about 200 hours. In one embodiment, the biologic composition provides a mean Tₘₐₓ of from about 30 hours to about 120 hours. In one embodiment, the biologic composition provides a mean Tₘₐₓ of from about 60 hours to about 200 hours. In one embodiment, the biologic composition provides a mean Tₘₐₓ of from about 1 day to about 7 days. In one embodiment, the biologic composition provides a mean Tₘₐₓ of from about 6 days to about 15 days. In one embodiment, the biologic composition provides a mean Tₘₐₓ of from about 4 days to about 8 days. In one embodiment, the biologic composition provides a mean Tₘₐₓ of from about 1 day to about 5 days. In one embodiment, the biologic composition provides a mean Tₘₐₓ of from about 2 days to about 6 days. In one embodiment, the biologic composition provides a mean Tₘₐₓ of from about 3 days to about 8 days.

In some embodiments, the biologic composition, when applied to the skin surface of the patient, provides a mean terminal half-life of from about from about 5 days to about 40 days. In some embodiments, the biologic composition, when applied to the skin surface of the patient, provides a mean terminal half-life of from about 5 to about 30 days, from about 5 days to about 20 days, from about 5 days to about 10 days, from about 7 days to about 40 days, from about 7 to about 30 days, from about 7 days to about 20 days, from about 7 days to about 10 days, from about 10 days to about 40 days, from about 10 to about 30 days, from about 10 days to about 20 days, from about 15 days to about 40 days, from about 15 to about 30 days, from about 15 days to about 20 days, from about 20 days to about 40 days, or from about 20 days to about 40 days. In one embodiment, the biologic composition provides a mean terminal half-life of from about 7 days to about 10 days. In one embodiment, the biologic composition provides a mean terminal half-life of from about 5 days to about 30 days.

In some embodiments, the biologic composition, when applied to the skin surface of the patient, provides a mean flux of from about 1 μg/cm²/hr to about 20 μg/cm²/hr. In some embodiments, the biologic composition, when applied to the skin surface of the patient, provides a mean flux of from about 1 μg/cm²/hr to about 15 μg/cm²/hr, from about 1 μg/cm²/hr to about 10 μg/cm²/hr, from about 1 μg/cm²/hr to about 5 μg/cm²/hr, from about 3 μg/cm²/hr to about 20 μg/cm²/hr, from about 3 μg/cm²/hr to about 15 μg/cm²/hr, from about 3 μg/cm²/hr to about 10 μg/cm²/hr, from about 3 μg/cm²/hr to about 5 g/cm²/hr, from about 5 μg/cm²/hr to about 20 μg/cm²/hr, from about 5 μg/cm²/hr to about 15 μg/cm²/hr, from about 5 μg/cm²/hr to about 10 μg/cm²/hr, from about 8 μg/cm²/hr to about 20 μg/cm²/hr, from about 8 μg/cm²/hr to about 15 μg/cm²/hr, from about 8 μg/cm²/hr to about 10 μg/cm²/hr, from about 10 μg/cm²/hr to about 20 μg/cm²/hr, from about 10 μg/cm²/hr to about 15 μg/cm²/hr, from about 12 μg/cm²/hr to about 20 μg/cm²/hr, from about 12 μg/cm²/hr to about 15 μg/cm²/hr, from about 15 μg/cm²/hr to about 20 μg/cm²/hr, or from about 18 μg/cm²/hr to about 20 μg/cm²/hr.

In other embodiments, the liquid composition that dries to a solid or semi-solid film comprises one or more topical anesthetic (“the anesthetic composition”). Examples of the topical anesthetic include, but are not limited to articaine, benzocaine, bupivacaine, butamben, chloroprocaine, cocaine, cyclomethycaine, dibucaine, dimethocaine, etidocaine, levobupivacaine, lidocaine, mepivacaine, novocaine, oxybuprocaine, pramoxine, piperocaine, prilocaine, proparacaine, propoxycaine, proxymetacaine, ropivacaine, tetracaine, and trimecaine. In one embodiment, the anesthetic is novocaine.

In some embodiments, the anesthetic composition comprises about 0.1% to about 15% by weight of one or more anesthetic. In some embodiments, the anesthetic composition comprises about 0.5% to about 15%, about 1% to about 15%, about 3% to about 15%, about 5% to about 15%, about 7% to about 15%, about 10% to about 15%, about 12% to about 15%, about 0.1% to about 12%, about 0.5% to about 12%, about 1% to about 12%, about 3% to about 12%, about 5% to about 12%, about 7% to about 12%, about 10% to about 12%, about 0.1% to about 10%, about 0.5% to about 10%, about 1% to about 10%, about 3% to about 10%, about 5% to about 10%, about 7% to about 10%, about 0.1% to about 5%, about 0.5% to about 5%, about 1% to about 5%, about 3% to about 5%, about 0.1% to about 3%, about 0.5% to about 3%, about 1% to about 3%, about 0.5% to about 1%, or about 0.1% to about 0.5% by weight of one or more anesthetic.

In some embodiments, the anesthetic composition, when applied to the skin surface of the patient, provides a mean Cₘₐₓ of from about 1 ng/ml to about 200 ng/ml. In some embodiments, the anesthetic composition, when applied to the skin surface of the patient, provides a mean Cₘₐₓ of from about 1 ng/ml to about 180 ng/ml, from about 1 ng/ml to about 130 ng/ml, from about 1 ng/mL to about 130 ng/mL, from about 1 ng/ml to about 100 ng/ml, from about 1 ng/mL to about 70 ng/ml, from about 1 ng/ml to about 50 ng/ml, from about 1 ng/mL to about 20 ng/ml, from about 1 ng/mL to about 10 ng/ml, from about 1 ng/ml to about 5 ng/mL, from about 10 ng/ml to about 200 ng/mL, from about 10 ng/ml to about 180 ng/ml, from about 10 ng/mL to about 130 ng/ml, from about 10 ng/ml to about 100 ng/mL, from about 10 ng/mL to about 70 ng/ml, from about 10 ng/ml to about 50 ng/mL, from about 10 ng/mL to about 20 ng/ml, from about 50 ng/ml to about 200 ng/mL, from about 50 ng/ml to about 180 ng/mL, from about 50 ng/ml to about 130 ng/ml, from about 50 ng/mL to about 100 ng/ml, from about 50 ng/ml to about 70 ng/mL, from about 100 ng/mL to about 200 ng/ml, from about 100 ng/ml to about 180 ng/ml, from about 100 ng/ml to about 130 ng/mL, from about 150 ng/ml to about 200 ng/ml, or from about 150 ng/ml to about 180 ng/mL.

In some embodiments, the anesthetic composition, when applied to the skin surface of the patient, provides a mean Tₘₐₓ of from about 1 minute to about 3 hours. In some embodiments, the anesthetic composition, when applied to the skin surface of the patient, provides a mean Tₘₐₓ of from about 5 minutes to about 3 hours, from about 15 minutes to about 3 hours, from about 30 minutes to about 3 hours, from about 45 minutes to about 3 hours, from about 1 hour to about 3 hours, from about 1.5 hours to about 3 hours, from about 2 hours to about 3 hours, from about 2.5 hours to about 3 hours, from about 1 minute to about 2.5 hours, from about 5 minutes to about 2.5 hours, from about 15 minutes to about 2.5 hours, from about 30 minutes to about 2.5 hours, from about 45 minutes to about 2.5 hours, from about 1 hour to about 2.5 hours, from about 1.5 hours to about 2.5 hours, from about 2 hours to about 2.5 hours, from about 1 minute to about 2 hours, from about 5 minutes to about 2 hours, from about 15 minutes to about 2 hours, from about 30 minutes to about 2 hours, from about 45 minutes to about 2 hours, from about 1 hour to about 2 hours, from about 1.5 hours to about 2 hours, from about 1 minute to about 1.5 hours, from about 5 minutes to about 1.5 hours, from about 15 minutes to about 1.5 hours, from about 30 minutes to about 1.5 hours, from about 45 minutes to about 1.5 hours, from about 1 hour to about 1.5 hours, from about 1 minute to about 1 hour, from about 5 minutes to about 1 hour, from about 15 minutes to about 1 hour, from about 30 minutes to about 1 hour, from about 45 minutes to about 1 hour, from about 1 minute to about 45 minutes, from about 5 minutes to about 45 minutes, from about 15 minutes to about 45 minutes, from about 30 minutes to about 45 minutes, from about 1 minute to about 30 minutes, from about 5 minutes to about 30 minutes, from about 15 minutes to about 30 minutes, from about 1 minute to about 15 minutes, from about 5 minutes to about 15 minutes, or from about 1 minute to about 5 minutes.

In some embodiments, the anesthetic composition, when applied to the skin surface of the patient, provides a mean flux of from about 1 μg/cm²/hr to about 20 μg/cm²/hr. In some embodiments, the anesthetic composition, when applied to the skin surface of the patient, provides a mean flux of from about 1 μg/cm²/hr to about 15 μg/cm²/hr, from about 1 μg/cm²/hr to about 10 μg/cm²/hr, from about 1 g/cm²/hr to about 5 μg/cm²/hr, from about 3 μg/cm²/hr to about 20 μg/cm²/hr, from about 3 μg/cm²/hr to about 15 μg/cm²/hr, from about 3 μg/cm²/hr to about 10 μg/cm²/hr, from about 3 μg/cm²/hr to about 5 μg/cm²/hr, from about 5 μg/cm²/hr to about 20 μg/cm²/hr, from about 5 μg/cm²/hr to about 15 μg/cm²/hr, from about 5 μg/cm²/hr to about 10 μg/cm²/hr, from about 8 μg/cm²/hr to about 20 μg/cm²/hr, from about 8 μg/cm²/hr to about 15 μg/cm²/hr, from about 8 μg/cm²/hr to about 10 μg/cm²/hr, from about 10 μg/cm²/hr to about 20 μg/cm²/hr, from about 10 μg/cm²/hr to about 15 μg/cm²/hr, from about 12 μg/cm²/hr to about 20 μg/cm²/hr, from about 12 μg/cm²/hr to about 15 μg/cm²/hr, from about 15 μg/cm²/hr to about 20 μg/cm²/hr, or from about 18 μg/cm²/hr to about 20 μg/cm²/hr.

In some embodiments, the anesthetic composition, when applied to the skin surface of the patient, provides a mean time for onset of action of from about 1 minute to about 2 hours. In some embodiments, the anesthetic composition, when applied to the skin surface of the patient, provides a mean time for onset of action of from about 1 minute to about 1.5 hours, from about 1 minute to about 1 hour, from about 1 minute to about 0.5 hours, from about 1 minute to about 15 minutes, from about 1 minute to about 10 minutes, from about 1 minute to about 5 minutes, from about 3 minutes to about 2 hours, from about 3 minutes to about 1.5 hours, from about 3 minutes to about 1 hour, from about 3 minutes to about 0.5 hours, from about 3 minutes to about 15 minutes, from about 3 minutes to about 10 minutes, from about 3 minutes to about 5 minutes, from about 5 minutes to about 2 hours, from about 5 minutes to about 1.5 hours, from about 5 minutes to about 1 hour, from about 5 minutes to about 0.5 hours, from about 5 minutes to about 15 minutes, from about 5 minutes to about 10 minutes, from about 10 minutes to about 2 hours, from about 10 minutes to about 1.5 hours, from about 10 minutes to about 1 hour, from about 10 minutes to about 0.5 hours, from about 10 minutes to about 15 minutes, from about 15 minutes to about 2 hours, from about 15 minutes to about 1.5 hours, from about 15 minutes to about 1 hour, from about 15 minutes to about 0.5 hours, from about 0.5 hours to about 2 hours, from about 0.5 hours to about 1.5 hours, from about 0.5 hours to about 1 hour, from about 1 hour to about 1.5 hours, from about 1 hour to about 2 hours, or from about 1.5 hours to about 2 hours.

In some embodiments, the liquid composition consists essentially of silicone gel that dries to a solid or semi-solid film. In some embodiments, the silicone gel consists essentially of about 2% to about 20%, about 5% to about 20%, about 10% to about 20%, about 15% to about 20%, about 2% to about 15%, about 5% to about 15%, about 10% to about 15%, about 2% to about 10%, about 5% to about 10%, or about 2% to about 5% by weight of silicone gel. In one embodiment, the silicone composition consists essentially of about 5% to about 15% by weight of silicone gel.

### Methods of Treatment

In some embodiments, the liquid compositions are used to treat a skin disorder, or for the prevention or reduction of scars. In certain embodiments, the liquid compositions are used to stimulate collagenase production in a patient suffering from a skin condition by applying a liquid composition comprising about 1% to about 10% by weight of a steroid to an area of affected skin surface of the patient. In certain embodiments, the skin condition is hypertrophic scars. In other embodiments, the skin condition is keloid scars. In still other embodiments, the condition is a combination of hypertrophic scars and keloid scars.

In certain embodiments, the liquid compositions are used to stimulate procollagenase production in a patient having one or more scars by applying a liquid composition comprising about 1% to about 10% by weight of a steroid to an area of affected skin surface of the patient. In some embodiments, the skin condition is hypertrophic scars. In other embodiments, the skin condition is keloid scars. In still other embodiments, the condition is a combination of hypertrophic scars and keloid scars.

In certain embodiments, the liquid compositions are used to stimulate collagenase and/or procollagenase production in a patient who suffers from scars that have recently formed as the result of accidental skin trauma, e.g. cuts, bruises, burns, or due to surgical procedures. In certain embodiments, the scars should be healed, i.e. re-epithelized such that the exterior dermis layer of the scar is intact. The patient can be treated hours to several months after the trauma depending on the extent of the wound and the vascularity of the area wounded.

In certain embodiments, the liquid compositions are used to stimulate collagenase and/or procollagenase production in a patient who suffers from scars that have formed for a relatively long period of time, such as hypertrophic scars.

In one embodiment, the procollagenase production is stimulated by at least about 150% to about 500% after 48 hours of applying the steroid composition to the patient.

In one embodiment, the collagenase production is stimulated by at least about 150% to about 500% after 48 hours of applying the steroid composition to the patient.

In some embodiments, the liquid compositions are used to treat a skin disorder (e.g., psoriasis, eczema, acne) or to prevent, reduce, lessen or alleviate discomfort or pain associated with certain medical procedures. In certain embodiments, the liquid compositions are used to treat a patient having a skin condition that is an inflammatory skin disorder. In certain embodiments, the liquid compositions are administered to a patient in need of pain management prior to a medical procedure.

Examples of inflammatory skin disorders that can be treated using the liquid compositions include, but are not limited to, acne, cold sore, blister, hives, actinic keratosis, rosacea, carbuncle, allergy, eczema, psoriasis, cellulitis, measles, basal cell carcinoma, squamous cell carcinoma, melanoma, lupus, contact dermatitis, vitiligo, warts, Human papillomaviruses (HPV) related lesions, chickenpox, seborrheic eczema, keratosis pilaris, ringworm, melasma, impetigo, rashes from bacterial or fungal infections, rashes from allergic reactions, and skin cancer. In certain embodiments, the liquid composition is used to treat a patient with psoriasis or eczema.

In some embodiments, the composition is applied to the affected area using any suitable applicator (e.g., a brush, roll, squeeze tube, sprayer or eye dropping apparatus). In some embodiments, the liquid composition is a relatively low or high viscous liquid which can be applied directly and accurately onto the affected area and does not require the application of additional pressure or rubbing.

In some embodiments, the area of affected skin surface to which the liquid composition is applied is from about 1 cm² to about 1000 cm². In certain embodiments, the area of affected skin surface to which the liquid composition is applied is from about 1 cm² to about 500 cm², from about 1 cm² to about 300 cm², from about 1 cm² to about 200 cm², from about 1 cm² to about 100 cm², from about 1 cm² to about 50 cm², from about 1 cm² to about 25 cm², from about 1 cm² to about 10 cm², or from about 1 cm² to about 5 cm². In one embodiment, the area of affected skin surface is from about 1 cm² to about 500 cm².

In some embodiments, the solid or semi-solid film formed by the composition is kept on the skin surface for from 1 to 7 days, from 1 to 5 days, from 1 to 3 days, from 3 to 7 days, from 3 to 5 days, or from 5 to 7 days, from 1 to 3 weeks, from 1 to 2 weeks, from 2 to 3 weeks, from 3 to 6 months, from 3 to 5 months, from 3 to 4 months, from 4 to 5 months, or from 5 to 6 months. The composition can be reapplied as needed if the solid or semi-solid film peels off the skin area. In some embodiments, the solid or semi-solid film is kept on the skin surface for 2 to 7 days, 1 to 3 weeks, or from 3 to 6 months.

In some embodiments, the liquid composition is applied to the affected skin area multiple times daily. In some embodiments, the liquid composition is applied to the affected skin surface in a single daily dose. In some embodiments, the liquid composition is applied to the affected skin surface for from 1 to 7 times a week, from 1 to 4 times a week, from 1 to 2 times a week, from 2 to 7 times a week, from 2 to 4 times a week, from 2 to 5 times a week, from 3 to 7 times a week, from 3 to 5 times a week, from 4 to 7 times a week, from 4 to 5 times a week, or from 5 to 7 times a week, up to 1 to 3 weeks, or 3 to 6 months. In one embodiment, the liquid composition is applied to the affected skin surface from 1 to 7 times a week.

In some embodiments, the liquid composition is reapplied to the affected skin area 1 to 3 times per day.

In some embodiments, the amount of the liquid composition that is applied to the affected skin area is a daily dose from about 0.05 ml to about 10 ml. In certain embodiments, the amount of the liquid composition that is applied to the affected skin area is a daily dose from about 0.05 ml to about 5 ml, from about 0.05 ml to about 3 ml, from about 0.05 ml to about 1 ml, from about 0.05 ml to about 0.5 ml, from about 0.5 ml to about 10 ml, from about 0.5 ml to about 5 ml, from about 0.5 ml to about 3 ml, from about 0.5 ml to about 1 ml, from about 1 ml to about 10 ml, from about 1 ml to about 5 ml, from about 1 ml to about 3 ml, from about 3 ml to about 10 ml, from about 3 ml to about 5 ml, from about 5 ml to about 10 ml, from about 5 ml to about 8 ml, or from about 7 ml to about 10 ml. In certain embodiments, the amount of liquid composition that is applied to the affected skin area is a daily dose of about 0.05 ml, about 0.1 ml, about 0.5 ml, about 1 ml, about 2 ml, about 3 ml, about 4 ml, about 5 ml, about 6 ml, about 7 ml, about 8 ml, about 9 ml, or about 10 ml. In one embodiment, the amount of the liquid composition that is applied to the affected skin area is from about 0.05 ml to about 5 ml.

In certain embodiments, the amount of the active ingredient that is applied to the affected skin area is a daily dose from about 0.1 mg to about 10 mg, from about 0.1 mg to about 5 mg, from about 0.1 mg to about 3 mg, from about 0.1 mg to about 1 mg, from about 0.1 mg to about 0.5 mg, from 0.5 mg to about 10 mg, from about 0.5 to about 5 mg, from about 0.5 mg to about 3 mg, from about 0.5 mg to about 1 mg, from about 1 mg to about 10 mg, from about 1 mg to about 5 mg, from about 1 mg to about 3 mg, from about 3 mg to about 10 mg, from about 3 mg to about 7 mg, from about 3 mg to about 5 mg, from about 5 mg to about 10 mg, from about 5 mg to about 7 mg, from about 7 mg to about 10 mg, from about 0.05 mg to about 15 mg, from about 0.05 mg to about 10 mg, from about 0.05 mg to about 5 mg, from about 0.05 mg to about 1 mg, from about 0.05 mg to about 0.5 mg, from about 0.1 mg to about 20 mg, from about 0.1 mg to about 15 mg, from about 0.5 mg to about 20 mg, from about 0.5 mg to about 15 mg, from about 1 mg to about 20 mg, from about 1 mg to about 15 mg, from about 3 mg to about 20 mg, from about 3 mg to about 15 mg, from about 5 mg to about 20 mg, from about 5 mg to about 15 mg, from about 7 mg to about 20 mg, from about 7 mg to about 15 mg, from about 10 mg to about 20 mg, from about 10 mg to about 15 mg, from about 15 mg to about 20 mg, from about 5 mg to about 1000 mg, from about 5 mg to about 500 mg, from about 5 mg to about 100 mg, from about 5 mg to about 50 mg, from about 10 mg to about 1000 mg, from about 10 mg to about 500 mg, from about 10 mg to about 100 mg, from about 10 mg to about 50 mg, from about 50 to about 1000 mg, from about 50 to about 500 mg, from about 50 mg to about 100 mg, from about 100 mg to about 1000 mg, from about 100 mg to about 500 mg, or from about 500 mg to about 1000 mg. In certain embodiments, the dose is applied to the affected skin surface of the patient in a single daily dose. In certain embodiments, the dose is applied to the affected skin surface of the patient in multiple daily doses.

In those embodiments where the active ingredient is a steroid, the amount of the steroid that is applied to the affected skin area in a daily dose is from about 0.1 mg to about 10 mg. In certain embodiments, the amount of steroid that is applied to the affected skin area is a daily dose from about 0.1 mg to about 5 mg, from about 0.1 mg to about 3 mg, from about 0.1 mg to about 1 mg, from about 0.1 mg to about 0.5 mg, from 0.5 mg to about 10 mg, from about 0.5 to about 5 mg, from about 0.5 mg to about 3 mg, from about 0.5 mg to about 1 mg, from about 1 mg to about 10 mg, from about 1 mg to about 5 mg, from about 1 mg to about 3 mg, from about 3 mg to about 10 mg, from about 3 mg to about 7 mg, from about 3 mg to about 5 mg, from about 5 mg to about 10 mg, from about 5 mg to about 7 mg, or from about 7 mg to about 10 mg. In certain embodiments, the dose is applied to the affected skin surface of the patient in a single daily dose. In certain embodiments, the dose is applied to the affected skin surface of the patient in multiple daily doses.

In those embodiments where the active ingredient is a biologic, the amount of the biologic that is applied to the affected skin area in a daily dose is from about 0.05 mg to about 20 mg. In certain embodiments, the amount of the biologic that is applied to the affected skin area in a daily dose is from about 0.05 mg to about 15 mg, from about 0.05 mg to about 10 mg, from about 0.05 mg to about 5 mg, from about 0.05 mg to about 1 mg, from about 0.05 mg to about 0.5 mg, from about 0.1 mg to about 20 mg, from about 0.1 mg to about 15 mg, from about 0.1 mg to about 10 mg, from about 0.1 mg to about 5 mg, from about 0.1 to about 3 mg, from about 0.1 to about 1 mg, from about 0.1 mg to about 0.5 mg, from about 0.5 mg to about 20 mg, from about 0.5 mg to about 15 mg, from about 0.5 mg to about 10 mg, from about 0.5 mg to about 5 mg, from about 0.5 mg to about 3 mg, from about 0.5 mg to about 1 mg, from about 1 mg to about 20 mg, from about 1 mg to about 15 mg, from about 1 mg to about 10 mg, from about 1 mg to about 5 mg, from about 1 mg to about 3 mg, from about 3 mg to about 20 mg, from about 3 mg to about 15 mg, from about 3 mg to about 10 mg, from about 3 mg to about 7 mg, from about 3 mg to about 5 mg, from about 5 mg to about 20 mg, from about 5 mg to about 15 mg, from about 5 mg to about 10 mg, from about 5 mg to about 7 mg, from about 7 mg to about 20 mg, from about 7 mg to about 15 mg, from about 7 mg to about 10 mg, from about 10 mg to about 20 mg, from about 10 mg to about 15 mg, or from about 15 mg to about 20 mg. In certain embodiments, the dose is applied to the affected skin surface of the patient in a single daily dose. In certain embodiments, the dose is applied to the affected skin surface of the patient in multiple daily doses.

In those embodiments where the active ingredient is an anesthetic, the amount of the anesthetic that is applied to the affected skin area in a daily dose is from about 5 mg to about 1000 mg. In certain embodiments, the amount of the anesthetic that is applied to the affected skin area in a daily dose is from about 5 mg to about 500 mg, from about 5 mg to about 100 mg, from about 5 mg to about 50 mg, from about 5 mg to about 10 mg, from about 10 mg to about 1000 mg, from about 10 mg to about 500 mg, from about 10 mg to about 100 mg, from about 10 mg to about 50 mg, from about 50 to about 1000 mg, from about 50 to about 500 mg, from about 50 mg to about 100 mg, from about 100 mg to about 1000 mg, from about 100 mg to about 500 mg, from about 500 mg to about 1000 mg. In certain embodiments, the dose is applied to the skin surface of the patient in a single dose. In certain embodiments, the dose is applied to the skin surface of the patient in multiple doses.

In those embodiments where the active ingredient is an anesthetic, the anesthetic composition is applied on to the skin surface from 10 minutes to 3 hours prior to a procedure. In some embodiments, the composition is applied on the skin surface from 10 minutes to 2.5 hours, from 10 minutes to 2 hours, from 10 minutes to 1.5 hours, from 10 minutes to 1 hour, from 10 minutes to 30 minutes, from 30 minutes to 3 hours, from 30 minutes to 2.5 hours, from 30 minutes to 2 hours, from 30 minutes to 1.5 hours, from 30 minutes to 1 hour, from 1 hour to 3 hours, from 1 hour to 2.5 hours, from 1 hour to 2 hours, from 1 hour to 1.5 hours, from 1.5 hours to 3 hours, from 1.5 hours to 2.5 hours, from 1.5 hours to 2 hours, from 2 hours to 3 hours, from 2 hours to 2.5 hours, from 2.5 hours to 3 hours prior to a procedure. In some embodiments, the procedure is injection, vaccination, biopsy, endoscopy, acupuncture, mole removal, or general surgery, or a medical procedure that causes discomfort or pain to the patient.

### EXAMPLES

### Example 1

### Measurement of Thickness

The following test is performed on the liquid composition to measure the thickness of the dried composition.

200 μl of the composition is mixed with 5 μl of 1% eosin Y and painted onto a coverslip. Images are collected using a Zeiss LSM 510 confocal microscope on samples that are in their liquid form and subsequently on samples that are allowed to dry. The dye is excited with HeNe 543 nm laser and Z-stack images are scanned under 560 nm long-pass filter with Zeiss Plan-Apochromat 63×/1.4 Oil immersion lens at intervals of 0.4 μm. Images are processed and measured with ImageJ.

### Example 2

Measurement of Cₘₐₓ and Tₘₐₓ

The following tests are performed on the liquid composition to measure Cₘₐₓ and Tₘₐₓ.

In study I, 18 human volunteers are enrolled, and in study II, 36 human volunteers are enrolled. For both the studies, study population included healthy, non-smoking, non-drinking males and females (non-pregnant) between the ages of 18 to 45 years and with a body mass index of 18-30 kg/m². Subjects are screened by medical history, clinical laboratory tests, and physical and skin examination. Absence of pregnancy is evaluated by urine pregnancy test. Subjects not meeting the above said criteria are excluded from the study. Vital signs such as temperature, pulse rate, and blood pressure are assessed prior to each treatment and also after 6 and 12 h of the application of the composition. Throughout the duration of both studies, volunteers are continuously observed and questioned for the occurrence of any adverse events. Written consent is obtained from all participants prior to entry into the study.

The patients are randomized to be applied with the composition (3 to 10 mg/24 h of fluticasone furoate; 0.1 to 0.33 ml/24 h of the composition; 5 to 20 cm²). The composition is applied to clean, dry, non-oily, non-irritated, and non-recently shaved skin, on the lower/mid-back area. The application area selected is away from any significant fold/creases and at least 1 inch away from the spine. Blood samples of 7 mL are collected periodically at −1, 0, 1.5, 3, 4, 6, 8, 9, 10, 11, 12, 13, 14, 15, 16, 20, 24, 30, and 36 h of post-patch-application, and plasma drug concentrations are determined by a validated liquid chromatography-tandem mass spectrometry (LC-MS/MS) method. General adhesion of the film formed by the composition is analyzed at 12, 24, 48, and 72 h after application.

Blood samples are centrifuged and the plasma is stored at −20° C. until analysis by LC-MS/MS. Plasma concentrations of the pharmaceutically active ingredient are analyzed by fortifying a 50-μL sample aliquot with 20 μL of internal standard working solution. A 200-μL solution of 2.0% ammonium hydroxide is then added, vortexed, and centrifuged. Then, the organic layer is transferred to a clean tube and a 20-μL volume of this final extract is injected and analyzed via HPLC equipped with MS/MS detection.

From both studies, PK data of the composition is taken for further analysis. PK analysis for both studies is done by using Phoenix software version 6.3 (Pharsight™, Certara L.P.). The PK parameters such as peak plasma concentration (Cₘₐₓ), time to reach Cₘₐₓ (Tₘₐₓ), area under plasma concentration time curve from time zero to time of last measurable concentration (AUC₀₋ₜ), area under plasma concentration time curve from time zero to time infinity (AUC_{0-inf}), and terminal elimination half-life (t_{1/2}) are calculated for both studies. PK modeling is performed by using plasma drug concentration-time profile of intravenous bolus (50 mg dose) data obtained from the literature. Compartmental analysis is performed by using Phoenix version 6.3 (Pharsight™, Certara L.P.). PK Solver is used for non-compartmental analysis. Selection of the model is based on best fit approach and other statistical parameters.

### Example 3

### Systematic Absorption

The following test is performed on the liquid composition to measure systemic absorption of the pharmaceutically active ingredient.

The systematic absorption of the pharmaceutically active ingredient in the composition is estimated by a two-stage procedure: deconvolution followed by comparison of fraction drug absorbed in vivo (Fa) to the fraction of drug permeated in vitro (Fp). Percent in vitro permeated is calculated by Eq. (1). Percent in vivo absorbed profile is calculated by using NCA and NDC methods.

To measure the fraction of drug permeated in vitro (Fp), Franz diffusion cells are used to investigate the ex vivo skin permeation of fluticasone from the composition. Human cadaver skin (HCS) is used as the barrier. 0.3 ml of the liquid composition containing 3 mg of the pharmaceutically active ingredient is brushed on the surface of the HCS. The temperature at which the study is performed is 37±2° C. Preparation of skin for ex vivo permeation study is performed by thawing the skin in 0.9% NaCl for not more than 1 h at room temperature and cutting to appropriate Franz cell size (≈5 cm²). This is followed by transferring the piece of the skin to the Franz cell which is filled with pH 6.8 phosphate buffer to equilibration for around 30 min. The reservoir compartment contains 5.0 mL of phosphate buffer, pH 6.8, from which 0.3 mL is withdrawn periodically using autosampler and analyzed by validated HPLC method. The cumulative medium correction is made to determine the total amount of fluticasone furoate permeated at each time points. This experiment is repeated with three skin donors, and three diffusion cells are used at each time (n=3). % Cₜₙ is calculated using the following Eq. (1):

$$\% {Ct}_{n} = \frac{{C}_{t} \times V \times 100}{S \times {C}_{T}} \qquad ( 1 )$$

where % Cₜₙ=normalized value of cumulative percent drug permeated, Cₜ=concentration at time t, V=volume of dissolution medium, S=surface area of skin, and C_{T}=concentration at terminal time point.

In NDC method, the in vitro data which follows a two compartment pharmacokinetic model is used to calculate the unit impulse response (UIR) values. The obtained UIR values are used to deconvolute the plasma drug concentration-time profiles of the pharmaceutically active ingredient. NDC method analysis is done by using Phoenix version 6.4 (Pharsight™, Certara L.P). Equation (2) represents the percent in vivo permeation of the drug using numerical deconvolution method:

$$C ( t ) = \text{?} ( t - u ) {r}_{abs} ( u ) du \qquad ( 2 )$$

$$\text{?} \text{indicates text missing or illegible when filed}$$

where r_{abs} is absorption rate time course, C_{δ} represents the concentration-time profile resulting from an instantaneous absorption of a unit amount of drug which is typically from bolus intravenous injection or reference oral solution data, C₍ₜ₎ represents the plasma concentration versus time profiles of the tested formulations, and u is the variable of integration.

In NCA method, the percent in vivo permeated is estimated by using the following Eq. (3):

$$\% in vivo absorbed = \frac{{AUC}_{( 0 - t )} \times {K}_{el} \times {V}_{d}}{F \times D} \times 100 \qquad ( 3 )$$

where AUC₍₀₋ₜ₎=area under time curve from time point (0−t), Kₑₗ=elimination constant, V_{d}=volume of distribution, F=percent bioavailable from the formulation, and D=dose administered.

### Example 4

### Flux

The following test is performed to measure flux.

Abdominal skin of male Wistar rats that weigh 250±20 g is used for the permeation studies. The rat is sacrificed with ether and the hair on the abdomen is carefully removed using an electric clipper. Full-thickness skin samples are cut, removed, and washed with normal saline. Adhering fat and connective tissues are carefully removed with a blunt-ended forceps. Skin is observed for any damage.

Full-thickness skin is mounted on Franz diffusion cells (vertical; available diffusion area, 2.54 cm²; volume of receiver cell, 13 mL) with a water jacket (32±1° C.) to assess skin permeability. The stratum corneum side is facing upward into the donor compartment, and the dermal side is facing downward into the receptor compartment. The receiver cells are filled with distilled water and stirred by a magnetic bar to ensure adequate mixing and maintenance of sink conditions. After the experiment began, all of the solutions are sampled at 1, 2, 4, 6, 8, and 10 h, filtered with micropore film (pore diameter, 0.45 μm), and an equal volume of blank solution is immediately added. Each data point represents the average of five examinations.

The permeation of the composition assayed for 10 h is investigated and plots of the cumulative amount of permeated drug (μg/cm²) are plotted versus time. The transdermal flux (μg/cm²/h) is calculated from the steady-state part of the curve and T_{lag} by extrapolation of the linear portion to the x-axis.

### Example 5

### Measurement of the Stimulation of Procollagenase and Collagenase Production

The following test is performed on the liquid composition to measure the stimulation of procollagenase and collagenase production.

Using the Mattek EpiDerm FT (MatTek Corp, Ashland, MA) tissue model, 3 cell culture groups are treated with varying amounts of the composition. A control group, with no product application (0% surface area occlusion); a group with 30% surface area occlusion (30 μL product application); and a third group with 100% surface area occlusion (100 μL product application). The subnatant, or nutrient fluid, supporting these cell cultures is aspirated at intervals of 0, 24, 48, and 72 hours and immediately frozen to and maintained at −40° C.

Procollagenase release into the culture medium is assayed using FITC-labeled bovine type 1 collagen as a substrate. To determine latent procollagenase activity, 50 μL from each of the 12 subnatant specimens is diluted to 190 μL with 0.05 M Tris HCl buffer, pH 7.8, containing 0.15 M NaCl and 0.005 M CaCl₂. The samples are then activated with 10 μL of 20 mM 4-aminophenylmercuric acetate (APMA) at 35° C. for 60 minutes. To determine the levels of collagenase, an additional 50 μL from these 12 samples are not activated with APMA and are diluted to 200 μL with the same buffer. The 24 samples, APMA activated and non-APMA activated, are then reacted with 100 μg of FITC collagen at 35° C. for 2 hours. Degradation products of FITC collagen are then isolated with an extraction buffer and the fluorescence intensity (FI) of the supernatant samples is determined using a fluorometer at excitation/emission of 490 nm/520 nm. These levels are compared to the FI of 100 μg of denatured FITC collagen. One unit of collagenolytic activity is defined as the cleavage of 1 μg of collagen per minute. In this study, 100 μg of collagen is used as a substrate; therefore, collagenase activity is calculated using the following equation: [(FI sample−FI blank)×100 μg]÷[(FI control−FI buffer)×reaction time (min)×sample volume (mL)].

### Example 6

The following composition is prepared by adding the ingredients shown below to the film-forming polymer. In this instance, the film forming polymer is pyroxylin.
5 wt. % silicone gel (Dow Corning 556),
3 wt. % mometasone,
3.5 wt. % pyroxylin,
64 wt. % diethyl ether, and
24.5 wt. % ethanol.

The composition is tested for thickness, pharmacokinetics and stimulation of production levels of procollagenase and collagenase using the same methods as introduced in Examples 1 to 5.

To treat hypertrophic scars, 0.2 to 0.5 ml of the liquid composition (6 mg to 15 mg of mometasone) is brushed onto about 4 cm² to about 25 cm² of the clean, affected skin surface. The patient can choose to brush on 0.2 ml to 0.5 ml of the liquid composition in a single daily dose in the morning or split the dose into two applications in the morning and in the evening. The solid or semi-solid film formed by the liquid composition stays on the skin surface for at least 12 hours. The patient peels off the solid or semi-solid film each time before a new dose of the liquid composition is brushed on to the affected skin surface.

### Example 7

The following composition is prepared and used for the treatment of psoriasis. The composition is prepared as by adding the ingredients shown below to the film-forming polymer. In this instance, the film-forming polymer is pyroxylin.
2 to 10 wt. % mometasone furoate,
1 to 10 wt. % pyroxylin,
60 to 75 wt. % diethyl ether, and
20 to 30 wt. % ethanol.

The composition is tested for thickness, pharmacokinetics and stimulation of production levels of procollagenase and collagenase using the same methods as introduced in Examples 1 to 5.

To treat psoriasis, 0.1 ml to 0.5 ml of the liquid composition (2 mg to 10 mg of mometasone furoate) is brushed onto about 4 cm² to about 40 cm² of the clean, affected skin surface. The composition is applied to multiple affected skin areas as spot treatments. The patient can choose to brush on 0.1 ml to 0.5 ml of the liquid composition in a single daily dose in the morning or split the dose into two applications in the morning and in the evening. The solid or semi-solid film formed by the liquid composition stays on the skin surface for at least 12 hours. The patient peels off the solid or semi-solid film each time before a new dose of the liquid composition is brushed on to the affected skin surface.

### Example 8

The following composition is prepared and used for the treatment of keloid scars. The composition is prepared by adding the ingredients shown below to the film-forming polymer. In this instance, the film-forming polymer is pyroxylin.
0.1 to 9 wt. % silicone gel (Dow Corning 556),
3 wt. % mometasone
0.1 to 0.4 wt. % vitamin E
1 to 10 wt. % pyroxylin,
60 to 75 wt. % diethyl ether, and
20 to 30 wt. % ethanol.

The composition is tested for thickness, pharmacokinetics and stimulation of production levels of procollagenase and collagenase using the same methods as introduced in Examples 1 to 5.

To treat keloid scars, 0.2 to 0.5 ml of the liquid composition (6 mg to 15 mg of mometasone) is brushed onto about 4 cm² to about 25 cm² of the clean, affected skin surface. The patient can choose to brush on 0.2 ml to 0.5 ml of the liquid composition in a single daily dose in the morning or split the dose into two applications in the morning and in the evening. The solid or semi-solid film formed by the liquid composition stays on the skin surface for at least 12 hours. The patient peels off the solid or semi-solid film each time before a new dose of the liquid composition is brushed on to the affected skin surface.

### Example 9

The following composition is prepared and used for the treatment of eczema. The composition is prepared by adding the ingredients shown below to the film-forming polymer. In this instance, the film-forming polymer is pyroxylin.
0.1 to 9 wt. % silicone gel (Dow Corning 556),
1 to 20 wt. % polyethylene glycol,
2 wt. % clobetasol propionate,
1 to 10 wt. % pyroxylin,
60 to 75% wt. diethyl either, and
20 to 30 wt. % ethanol.

The composition is tested for thickness, pharmacokinetics and stimulation of production levels of procollagenase and collagenase using the same methods as introduced in Examples 1 to 5.

To treat eczema, 0.1 to 0.25 ml of the liquid composition is brushed onto about 4 cm² to about 25 cm² of the clean, affected skin surface. The composition is applied to multiple affected skin areas as spot treatments. The patient can choose to brush on 0.1 to 0.25 ml of the liquid composition in a single daily dose in the morning or split the dose into two applications in the morning and in the evening. The solid or semi-solid film formed by the liquid composition stays on the skin surface for at least 12 hours. The patient peels off the solid or semi-solid film each time before a new dose of the liquid composition is brushed on to the affected skin surface.

### Example 10

The following composition is prepared and used for the treatment of hypertrophic scars. The composition is prepared by adding the ingredients shown below to the film-forming polymer. In this instance, the film forming polymer is pyroxylin.
0.1 to 9 wt. % silicone gel (Dow Corning 556),
5 wt. % mometasone,
1 to 20 wt. % propylene glycol,
1 to 10 wt. % pyroxylin,
60 to 75 wt. % diethyl ether, and
20 to 30 wt. % ethanol.

The composition is tested for thickness, pharmacokinetics and stimulation of production levels of procollagenase and collagenase using the same methods as introduced in Examples 1 to 5.

To treat hypertrophic scars, 0.05 to 0.3 ml of the liquid composition is brushed onto about 4 cm² to about 10 cm² of the clean, affected skin surface. The patient can choose to brush on 0.05 ml to 0.3 ml of the liquid composition in a single daily dose in the morning or split the dose into two applications in the morning and in the evening. The solid or semi-solid film formed by the liquid composition stays on the skin surface for at least 12 hours. The patient peels off the solid or semi-solid film each time before a new dose of the liquid composition is brushed on to the affected skin surface.

### Example 11

The following composition is prepared and used for the treatment of scars. The composition is prepared by adding the ingredients shown below to the film-forming polymer. In this instance, the film forming polymer is pyroxylin.
8.5 wt. % silicone gel (Dow Corning 556),
1 wt. % mometasone,
3.5 wt. % pyroxylin,
63.5 wt. % diethyl ether, and
23.5 wt. % ethanol.

The composition is tested for thickness, pharmacokinetics and stimulation of production levels of procollagenase and collagenase using the same methods as introduced in Examples 1 to 5.

To treat scars, 0.7 to 1.5 ml of the liquid composition is brushed onto about 10 cm² to about 50 cm² of the clean, affected skin surface. The patient can choose to brush on 0.05 ml to 0.3 ml of the liquid composition in a single daily dose in the morning or split the dose into two applications in the morning and in the evening. The solid or semi-solid film formed by the liquid composition stays on the skin surface for at least 12 hours. The patient peels off the solid or semi-solid film each time before a new dose of the liquid composition is brushed on to the affected skin surface.

### Example 12

The following composition is prepared and used for the treatment of keloid scars. The composition is prepared by adding the ingredients shown below to the film-forming polymer. In this instance, the film-forming polymer is pyroxylin.
4 wt. % silicone gel (Dow Corning 556),
9 wt. % mometasone
3 wt. % pyroxylin,
61 wt. % diethyl ether, and
23 wt. % ethanol.

The composition is tested for thickness, pharmacokinetics and stimulation of production levels of procollagenase and collagenase using the same methods as introduced in Examples 1 to 5.

To treat keloid scars, 0.05 to 0.2 ml of the liquid composition is brushed onto about 2 cm² to about 10 cm² of the clean, affected skin surface. The patient can choose to brush on 0.05 ml to 0.2 ml of the liquid composition in a single daily dose in the morning or split the dose into two applications in the morning and in the evening. The solid or semi-solid film formed by the liquid composition stays on the skin surface for at least 12 hours. The patient peels off the solid or semi-solid film each time before a new dose of the liquid composition is brushed on to the affected skin surface.

### Example 13

The following composition is prepared by adding the ingredients shown below to the film-forming polymer. In this instance, the film-forming polymer is pyroxylin.
2 to 10 wt. % etanercept,
1 to 10 wt. % pyroxylin,
60 to 75 wt. % diethyl ether, and
20 to 30 wt. % ethanol.

The composition is tested for thickness and pharmacokinetics using the same methods as introduced in Examples 1 to 4.

The composition is further tested for its efficacy in treating inflammatory skin diseases. First, the binding capacity of etanercept with tumor necrosis factor alpha (TNFα) is tested. TNFα is a cell signaling protein involved in systemic inflammation and is one of the cytokines that make up the acute phase reaction. The primary role of TNFα is in the regulation of immune cells. The concentration of etanercept is determined by bicinchoninic acid assay (BCA) (Pierce™ BCA protein Assay Kit, Thermo Scientific, Waltham, MA, USA), and then all samples are adjusted to 10 ng/ml. The binding affinity of etanercept for TNFα is determined by a commercially available sandwich ELISA that incorporates plate bound TNFα (Sanquin, Diagnostic Services, Amsterdam, Netherlands). Results are expressed as the samples TNFα binding as a percentage of fresh ETR TNFα binding.

Next, the efficacy of the composition for treating inflammatory skin diseases is demonstrated by an in vitro experiment using the previously established model of TNFα mediated skin inflammation. To induce an inflammation-like state, normal skin equivalents are supplemented with TNFα. Human skin equivalents are prepared from primary human keratinocytes and fibroblasts. Primary skin cells are derived from normal human skin with written consent. To induce skin inflammation, 20 ng/mL recombinant TNFα (eBiosdence, Hatfield, UK) is supplemented into the skin equivalents growth media on days 10 and 12 of cultivation. The etanercept composition (35 μg/cm² of etanercept; 1.75 ml/cm² of the composition) is applied directly to the surface of skin equivalents 24 h after TNFα treatment (days 11 and 13) and exposed to a temperature gradient to simulate the natural temperature gradient of human skin (32 to 37° C. over 3 h). On day 14, skin equivalents are halved and prepared separately for western blotting and immunohistochemistry.

Western blotting of the skin samples is performed to measure the protein content levels of TNFα, TSLP, and ICAM1. Halved skin equivalents are lysed in RIPA buffer (supplemented with protease and phosphatase inhibitors) according to standard procedure. Protein content is quantified by BCA assay. Samples (15 μg protein) are then heated in SDS-PAGE buffer and separated by electrophoresis through a polyacrylamide gel (10%). Gels are blotted onto nitrocellulose membranes, blocked (5% skimmed-milk powder), exposed to primary antibodies (overnight, 4° C., washed, incubated with horseradish peroxidise conjugated secondary antibodies (1 h, room temperature) and washed again. Blots are developed with ECL reagent (SignalFire™, Cell Signaling, Frankfurt/Main, Germany) and imaged by a PXi/PXi Touch gel imaging system (Syngene, Cambridge, UK). Antibodies are used at the following concentrations: 1:1000 anti-TNFα, 1:1000 anti-TSLP, anti-ICAM1 1:2000, 1:500 anti-IgG Rabbit conjugated to horseradish peroxidase.

Immuno-histochemical staining visually shows the level of etanercept that permeates the skin in the skin equivalents topically treated with the etanercept composition. Halved skin equivalents are submerged in tissue freezing media, and flash frozen. Samples are subsequently cut into cross sections (8 μm) on a cryotome (Leica, PLACE) against the direction of application (i.e., deep to superficial). Skin sections are fixed using a 4% formaldehyde solution, washed with PBS containing 0.0025% BSA and 0.025% Tween 20 and blocked with goat serum (1:20 in PBS). Subsequently, skin sections are incubated overnight at 4° C. with primary antibodies. After washing, secondary antibodies are added for 1 h at room temperature, and finally skin sections are covered with anti-fading mounting medium. Images are analyzed under a fluorescence microscope (BZ-8000, objectives 20×/0.75, zoom lOx, Plan-Apo, DIC N2, Keyence, Neu-Isenburg, Germany). Antibodies are used at the following concentrations: 1:500 anti-TNFα, 1:500 anti-IgG Rabbit conjugated to Alexa 594.

Monocyte derived Langerhans cells (MoLCs) are used to determine how the etanercept composition interferes with immune system. Upon immunological activation by etanercept, immature MoLCs elevate surface expression of CD86 and CD83. Expressions of these markers are measured by flow cytometric analysis as an assay of immunogenicity. MoLCs are generated from isolated human monocytes. After seven days of cultivation, MoLCs are collected and characterized by surface expression of CD1a and CD207. Afterwards, MoLCs are seeded into 24-well plates (2.5×10⁵ cells/well) and incubated with the etanercept composition for 24 h. The immunogenic effects of the etanercept composition are determined by the cell-surface expression CD83 and CD86. Additionally, cytotoxicity is measured by staining the cells with 7-Aminoactinomycin D (7-AAD) (Sigma-Aldrich, St. Louis, USA). Surface receptor expression and 7-AAD penetration are assessed by flow cytometry (FACSCanto II, BD Biosciences, Heidelberg, Germany) and the resulting data is analyzed by FlowJo software (Treestar, Ashland, USA).

### Example 14

The following composition is prepared by adding the ingredients shown below to the film-forming polymer. In this instance, the film forming polymer is pyroxylin.
3-10 wt. % adalimumab,
1 to 10 wt. % pyroxylin,
40 to 50 wt. % polyoxyethylene lauryl ether, and
20 to 30 wt. % ethanol.

The composition is tested for thickness and pharmacokinetics using the same methods as introduced in Examples 1 to 4. The composition is further tested for its efficacy in treating inflammatory skin diseases and interfering with the immune system using similar methods as introduced in Example 13.

### Example 15

The following composition is prepared by adding the ingredients shown below to the film-forming polymer. In this instance, the film forming polymer is pyroxylin.
8-15 wt. % infliximab,
1 to 10 wt. % pyroxylin,
60 to 75 wt. % diethyl ether, and
20 to 30 wt. % ethanol.

The composition is tested for thickness and pharmacokinetics using the same methods as introduced in Examples 1 to 4. The composition is further tested for its efficacy in treating inflammatory skin diseases and interfering with the immune system using similar methods as introduced in Example 13.

### Example 16

The following composition is prepared by adding the ingredients shown below to the film-forming polymer. In this instance, the film forming polymer is pyroxylin.
0.5-10 wt. % secukinumab,
1 to 10 wt. % pyroxylin,
40 to 50 wt. % polyoxyethylene lauryl ether, and
20 to 30 wt. % ethanol.

The composition is tested for thickness and pharmacokinetics using the same methods as introduced in Examples 1 to 4. The composition is further tested for its efficacy in treating inflammatory skin diseases and interfering with the immune system using similar methods as introduced in Example 13.

### Example 17

The following composition is prepared by adding the ingredients shown below to the film-forming polymer. In this instance, the film forming polymer is pyroxylin.
4.5 wt. % infliximab,
1 to 10 wt. % pyroxylin,
60 to 75 wt. % diethyl ether, and
20 to 30 wt. % ethanol.

The composition is tested for thickness and pharmacokinetics using the same methods as introduced in Examples 1 to 4. The composition is further tested for its efficacy in treating inflammatory skin diseases and interfering with the immune system using similar methods as introduced in Example 13.

### Example 18

The following composition is prepared by adding the ingredients shown below to the film-forming polymer. In this instance, the film forming polymer is pyroxylin.
3 wt. % adalimumab,
1 to 10 wt. % pyroxylin,
40 to 50 wt. % polyoxyethylene lauryl ether, and
20 to 30 wt. % ethanol.

The composition is tested for thickness and pharmacokinetics using the same methods as introduced in Examples 1 to 4. The composition is further tested for its efficacy in treating inflammatory skin diseases and interfering with the immune system using similar methods as introduced in Example 13.

### Example 19

The following composition is prepared by adding the ingredients shown below to the film-forming polymer. In this instance, the film forming polymer is pyroxylin.
8 wt. % infliximab,
1 to 10 wt. % pyroxylin,
60 to 75 wt. % diethyl ether, and
20 to 30 wt. % ethanol.

The composition is tested for thickness and pharmacokinetics using the same methods as introduced in Examples 1 to 4. The composition is further tested for its efficacy in treating inflammatory skin diseases and interfering with the immune system using similar methods as introduced in Example 13.

### Example 20

The following composition is prepared by adding the ingredients shown below to the film-forming polymer. In this instance, the film forming polymer is pyroxylin.
3 wt. % adalimumab,
4 wt. % pyroxylin,
72 wt. % polyoxyethylene lauryl ether, and
21 wt. % ethanol.

The composition is tested for thickness and pharmacokinetics using the same methods as introduced in Examples 1 to 4. The composition is further tested for its efficacy in treating inflammatory skin diseases and interfering with the immune system using similar methods as introduced in Example 13.

### Example 21

The following composition is prepared by adding the ingredients shown below to the film-forming polymer. In this instance, the film forming polymer is pyroxylin.
8 wt. % infliximab,
5 wt. % pyroxylin,
63 wt. % diethyl ether, and
24 wt. % ethanol.

The composition is tested for thickness and pharmacokinetics using the same methods as introduced in Examples 1 to 4. The composition is further tested for its efficacy in treating inflammatory skin diseases and interfering with the immune system using similar methods as introduced in Example 13.

### Example 22

The following composition is prepared as by adding the ingredients shown below to the film-forming polymer. In this instance, the film forming polymer is pyroxylin.
15 wt. % silicone gel (Dow Corning 556),
3.5 wt. % pyroxylin,
58 wt. % diethyl ether, and
23.5 wt. % ethanol.

The composition is tested for thickness and pharmacokinetics using the same methods as introduced in Examples 1 to 5.

To treat hypertrophic scars, 0.2 ml of the liquid composition is brushed to about 25 cm² of the clean, affected skin surface. The patient can choose to brush on 0.2 ml of the liquid composition in a single daily dose in the morning or split the dose into two applications in the morning and in the evening. The solid or semi-solid film formed by the liquid composition stays on the skin surface for at least 12 hours. The patient peels off the solid or semi-solid film before each time a new dose of the liquid composition is brushed on to the affected skin surface.

### Example 23 Vasoconstriction Testing

The objective of the following studies is to evaluate transpore-delivery of various drugs.

### Example 23a Cortisone Vasoconstriction Test

Materials used in the cortisone formulation are listed below:
23.76 wt. % Nitrocellulose-CAS #9004-70-0
37.62 wt. % Diethyl Ether-CAS #60-29-7
37.62 wt. % Ethyl Alcohol-CAS #64-1-5
1.00 wt. % Cortisone-CAS #53-06-5.

Experimental Procedure: the biological effect of transpore-delivered cortisone was tested utilizing the FDA recommended method developed by Mckenzie and Stoughton to assess the vasoconstriction effects of 1% cortisone (Mckenzie A W, Stoughton R B., Method for Comparing Percutaneous Absorption of Steroids, Arch. Dermatol. 86, 608 (1962)). A Minolta Chroma Meter (CR-300) was used to measure the blanching of the skin. An area on the ventral area of the subject's forearm was selected due to the scarcity of hair and its relative uniform skin tone. Baseline lightness was measured in both test and control areas. Following the baseline measurements, the nitrocellulose film (without cortisone) was brushed on and allowed to dry on three (3) sites. The nitrocellulose formulation with cortisone was applied to three (3) other test sites. After one (1) hour, the film was removed and the blanching was measured.

The mean percent change from baseline in skin blanching for the nitrocellulose film alone was −0.81±1.01 percent, and the mean percent change from baseline for the nitrocellulose with 1% cortisone was 1.38±0.85. Statistical analysis was performed using a two-tailed t-test. The results indicated that there was a significant difference between the nitrocellulose film alone and that containing the steroid cortisone (p=0.045). The result from the skin blanching test verifies that cortisone is absorbed via transpore delivery. The significantly different mean percent changes from baseline between the nitrocellulose with or without 1% cortisone indicate that cortisone effectively penetrates the skin which usually is a barrier to drug penetration.

Conclusion: these results demonstrate effective transpore-delivery of a biologically-active steroid.

### Example 23b Etanercept Vasoconstriction Test

Materials used in the cortisone formulation are listed below:
23.70 wt. % Nitrocellulose-CAS #9004-70-0
37.53 wt. % Diethyl Ether-CAS #60-29-7
37.53 wt. % Ethyl Alcohol-CAS #64-1-5
1.25 wt. % Etanercept-CAS #185243-69-0.

Experimental Procedure: the biological effect of transpore-delivered etanercept was tested utilizing the FDA recommended method developed by Mckenzie and Stoughton to assess the vasoconstriction effects of 1.25% etanercept (Mckenzie A W, Stoughton R B., Method for Comparing Percutaneous Absorption of Steroids, Arch. Dermatol. 86, 608 (1962)). A Minolta Chroma Meter (CR-300) was used to measure the blanching of the skin. An area on the ventral area of the subject's forearm was selected due to the scarcity of hair and its relative uniform skin tone. Baseline lightness was measured in both test and control areas. Following the baseline measurements, the nitrocellulose film (without etanercept) was brushed on and allowed to dry on four (4) sites. The nitrocellulose formulation with etanercept was applied to four (4) other test sites, and 1% OTC hydrocortisone cream (CVS 6870032439—Exp. Jun. 20, 2021) was applied to four (4) additional sites. After one (1) hour, the film was removed and the blanching was measured.

The mean percent change from baseline in skin blanching for the nitrocellulose film alone was 0.42±1.51 percent, the mean percent change from baseline for the nitrocellulose with 1.25% etanercept was 3.35±2.01 percent, and the mean percent change from baseline for the 1% OTC hydrocortisone cream was 3.82±2.04 percent. Statistical analysis was performed using an ANOVA. Since the ANOVA analysis indicated that there was a significant among group difference (p=0.02), pairwise comparisons were performed using a two-tailed t-test. The results indicated that there was a significant difference between the nitrocellulose film with 1.25% etanercept and the nitrocellulose film alone (p=0.02) and between the 1% OTC hydrocortisone cream and the nitrocellulose film alone (p=0.02). There were no significant differences between the nitrocellulose film with 1.25% etanercept and the 1% OTC hydrocortisone cream (p=0.91). The result from the skin blanching test verifies that etanercept is absorbed via transpore delivery. The significantly different mean percent changes from baseline between the nitrocellulose with or without 1.25% etanercept indicate that etanercept effectively penetrates the skin which usually is a barrier to drug penetration.

Conclusion: these results demonstrate effective transpore-delivery of a biological agent.

1. A method for transpore delivery of a steroid to a patient suffering from a skin condition, the method comprising applying a liquid composition comprising about 1% to about 10% by weight of said steroid to an area of affected skin surface of the patient,
said composition, when applied to the skin surface of the patient, achieves one or more of the following: (a) has a thickness of about 0.1 μm to about 10 μm in solid or semi-solid form, (b) forms a solid or semi-solid film, and (c) provides a mean Tₘₐₓ of from about 0.5 hours to about 8 hours,
wherein said composition seeps into skin pores in liquid form and creates a biomechanical integration with the interior of said skin pores in solid form, and
wherein said skin condition is selected from the group consisting of inflammatory skin conditions, hypertrophic scars, keloid scars, or a combination thereof.

2.-9. (canceled)

10. The method of claim 1, wherein said composition further comprises about 0% to about 9% by weight of silicone gel.

11. The method of claim 1, wherein said composition further comprises about 50% to about 99% by weight of pyroxylin, ether and alcohol.

12. The method of claim 1, wherein said skin condition is an inflammatory skin condition.

13. The method of claim 1, wherein said skin condition is hypertrophic scars.

14. The method of claim 1, wherein said skin condition is keloid scars.

15. The method of claim 1, wherein said steroid is selected from the group consisting of one or more of clobetasol propionate, flurandrenolide, betamethasone dipropionate, diflorasone diacetate, desoximetasone, halobetasol propionate, fluocinonide, mometasone furoate, mometasone, halcinonide, desoximetasone, fluticasone propionate, triamcinolone acetonide, hydrocortisone valerate, fluocinolone acetonide, prednicarbate, desonide, hydrocortisone, fluocinolone acetonide, hydrocortisone valerate, alclometasone dipropionate, and other pharmaceutically acceptable salts thereof.

16. The method of claim 1, wherein said steroid is mometasone or a pharmaceutically acceptable salt thereof.

17. The method of claim 1, wherein about 0.5 mg to about 10 mg of said steroid is applied on to the affected skin surface in a daily dose.

18. The method of claim 1, wherein about 0.05 ml to about 5 ml of said composition is applied on to the skin surface in a daily dose.

19.-21. (canceled)

22. A liquid composition comprising pyroxylin, ether, alcohol, and about 1% to about 10% by weight of steroid, said composition, when applied to an area of affected skin surface of a patient suffering from a skin condition, achieves one or more of the following: (a) has a thickness of about 0.1 μm to about 10 μm in solid form, (b) forms a solid or semi-solid film, and (c) provides a mean Tₘₐₓ of from about 0.5 hours to about 8 hours.

23. (canceled)

24. The composition of claim 22, wherein the steroid is mometasone or a pharmaceutically acceptable salt thereof.

25. The composition of claim 22, wherein said composition seeps into skin pores in liquid form and creates a biomechanical integration with the interior of said skin pores in solid form.

26.-29. (canceled)

30. A method of treating a patient suffering from a skin condition, the method comprising applying the composition of claim 22 to an area of affected skin surface of the patient,
wherein said area of skin surface is from about 1 cm² to about 500 cm², and wherein said skin condition is selected from the group consisting of inflammatory skin conditions, hypertrophic scars, and keloid scars, or a combination thereof.

31. The method of claim 30, wherein said skin condition is an inflammatory skin condition.

32. The method of claim 30, wherein said skin condition is hypertrophic scars.

33. The method of claim 30, wherein said skin condition is keloid scars.

34.-83. (canceled)

84. A liquid composition comprising about 0.001% to about 15% of an active ingredient by weight, about 0% to about 9% of silicone gel by weight, pyroxylin, ether, and alcohol, said composition, when applied to the skin surface of a patient, seeps into skin pores in liquid form and creates a biomechanical integration with the interior of said skin pores in solid form.

85.-86. (canceled)

87. The composition of claim 84, wherein said active ingredient is a steroid or an anesthetic.

88.-98. (canceled)
